(19) United States
(12) Patent Application Publication
(10) Pub. No.: US 2007/0072181 A1
(43) Pub. Date: Mar. 29, 2007

(54) Combination therapy for treating alphavirus infection and liver fibrosis

(76) Inventor: Lawrence M. Blatt, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/545,864

(22) PCT Filed: Feb. 26, 2004
(86) PCT No.: PCT/US04/05862

§ 371 Date: Jun. 29, 2006

Related U.S. Application Data
(60) Provisional application No. 60/451,316, filed on Feb. 28, 2003.

(51) Int. Cl. C12Q 1/70; C12Q 1/68

(52) U.S. Cl. 435/6; 435/5

(57) ABSTRACT

The present invention provides methods for treating alphavirus infections; methods of treating hepatitis C virus (HCV) infections; methods of treating West Nile virus infection; methods of reducing liver fibrosis; methods of increasing liver function in an individual suffering from liver fibrosis; methods of reducing the incidence of complications associated with HCV and cirrhosis of the liver; and methods of reducing viral load, or reducing the time to viral clearance, or reducing morbidity or mortality in the clinical outcomes, in patients suffering from viral infection. The methods generally involve administering effective amounts of an interferon receptor agonist and pirfenidone in combination therapy.

### FIELD OF THE INVENTION

The present invention is in the field of treatment of alphavirus infection

### BACKGROUND OF THE INVENTION

The family Alphaviridae includes influenza viruses, parainfluenza viruses, picornaviruses, polio virus, flaviviruses, e.g. yellow fever virus, the four serotypes of dengue virus, Japanese encephalitis virus, Tick-borne encephalitis virus, West Nile virus, hepatitis viruses, and many other disease causing viruses.

Hepatitis C virus is an illustrative example of the family of alphaviruses. Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

Anitiviral therapy of chronic hepatitis C has evolved rapidly over the last decade, with significant improvements seen in the efficacy of treatment. Nevertheless, even with combination therapy using pegylated IFN-α plus ribavirin, 40% to 50% of patients fail therapy, i.e., are nonresponders or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, this will result in a substantial increase in cirrhosis-related morbidity and mortality among patients infected between the years of 1965-1985.

Fibrosis occurs as a result of a chronic toxic insult to the liver, such as chronic hepatitis C virus (HCV) infection, autoimmune injury, and chronic exposure to toxins such as alcohol. Chronic toxic insult leads to repeated cycles of hepatocyte injury and repair accompanied by chronic inflammation. Over a variable period of time, abnormal extracellular matrix progressively accumulates as a consequence of the host's wound repair response. Left unchecked, this leads to increasing deposition of fibrous material until liver architecture becomes distorted and the liver's regenerative ability is compromised. The progressive accumulation of scar tissue within the liver finally results in the histopathologic picture of cirrhosis, defined as the formation of fibrous septae throughout the liver with the formation of micronodules.

There is a need in the art for methods of treating alphavirus infections in general, and HCV infection in particular. The present invention addresses this need, and provides related advantages.

### LITERATURE

U.S. Pat. Nos. 5,252,714; 5,382,657; 5,539,063; 5,559,213; 5,672,662; 5,747,646; 5,766,581; 5,792,834; 5,795,569; 5,798,232; 5,824,784; 5,834,594; 5,849,860; 5,928,636; 5,951,974; 5,595,732; 5,981,709; 6,005,075; 6,180,096; 6,250,469; 6,277,830. PCT Publication No. WO 99/37779. Chamov et al. (1994) Bioconj. Chem. 5:133-140; Harris et al. (2001) Clin. Pharmacokinet. 40:539-551; Reddy (2000) Ann. Pharmacother. 34:915-923; Reddy et al. (2002) Adv. Drug Deliv. Rev. 54:571-586. Pirfenidone (5-methyl-1-phenyl-2-(1H)-pyridone) and analogs thereof are described in, for example, U.S. Pat. Nos. 3,974,281; 5,310,562; 5,518,729; 5,716,632; and 6,090,822.

METAVIR (1994) Hepatology 20:15-20; Brunt (2000) Hepatol. 31:241-246; Alpini (1997) J. Hepatol. 27:371-380; Baroni et al. (1996) Hepatol. 23:1189-1199; Czaja et al. (1989) Hepatol. 10:795-800; Grossman et al. (1998) J. Gastroenterol. Hepatol. 13:1058-1060; Rockey and Chung (1994) J. Invest. Med. 42:660-670; Sakaida et al. (1998) J. Hepatol. 28:471-479; Shi et al. (1997) Proc. Natl. Acad. Sci. USA 94:10663-10668; Baroni et al. (1999) Liver 19:212-219; Lortat-Jacob et al. (1997) J. Hepatol. 26:894-903; Llorent et al. (1996) J. Hepatol. 24:555-563; U.S. Pat. No. 5,082,659; European Patent Application EP 294,160; U.S. Pat. No. 4,806,347; Balish et al. (1992) J. Infect. Diseases 166:1401-1403; Katayama et al. (2001) J. Viral Hepatitis 8:180-185; U.S. Pat. No. 5,082,659; U.S. Pat. No. 5,190,751; U.S. Pat. No. 4,806,347; Wandl et al. (1992) Br. J. Haematol. 81:516-519; European Patent Application No. 294,160; Canadian Patent No. 1,321,348; European Patent Application No. 276,120; Wandl et al. (1992) Sem. Oncol. 19:88-94; Balish et al. (1992) J. Infectious Diseases 166:1401-1403; Van Dijk et al. (1994) Int. J. Cancer 56:262-268; Sundmacher et al. (1987) Current Eye Res. 6:273-276; U.S. Pat. Nos. 6,172,046; 6,245,740; 5,824,784; 5,372,808; 5,980,884; published international patent applications WO 96/21468; WO 96/11953; Torre et al. (2001) J. Med. Virol. 64:455-459; Bekkering et al. (2001) J. Hepatol. 34:435-440; Zeuzem et al. (2001) Gastroenterol. 120:1438-1447; Zeuzem (1999) J. Hepatol. 31:61-64; Keeffe and Hollinger (1997) Hepatol. 26:101S-107S; Wills (1990) Clin. Pharmacokinet. 19:390-399; Heathcote et al. (2000) New Engl. J. Med. 343:1673-1680; Husa and Husova (2001) Bratisl. Lek. Listy 102:248-252; Glue et al. (2000) Clin. Pharmacol. 68:556-567; Bailon et al. (2001) Bioconj. Chem. 12:195-202; and Neumann et al. (2001) Science 282:103; Zalipsky (1995) Adv. Drug Delivery Reviews S. 16, 157-182; Mann et al. (2001) Lancet 358:958-965; Zeuzem et al. (2000) New Engl. J. Med. 343:1666-1672; U.S. Pat. Nos. 5,985,265; 5,908,121; 6,177,074; 5,985,263; 5,711,944; 5,382,657; and 5,908,121; Osborn et al. (2002) J. Pharmacol. Exp. Therap. 303:540-548; Sheppard et al. (2003) Nat. Immunol. 4:63-68; Chang et al. (1999) Nat. Biotechnol. 17:793-797; Adolf (1995) Multiple Sclerosis 1 Suppl. 1:S44-S47.

### SUMMARY OF THE INVENTION

The present invention provides methods for treating alphavirus infections; methods of treating hepatitis C virus (HCV) infections; methods of treating West Nile virus infection; methods of reducing liver fibrosis; methods of increasing liver function in an individual suffering from liver fibrosis; methods of reducing the incidence of complications associated with HCV and cirrhosis of the liver; and methods of reducing viral load, or reducing the time to viral clearance, or reducing morbidity or mortality in the clinical outcomes, in patients suffering from viral infection. The methods generally involve administering effective amounts of an interferon receptor agonist and pirfenidone (or a pirfenidone analog) in combination therapy.

### Features of the Invention

The invention features a method of treating alphaviral infection, generally involving administering to an individual an interferon receptor agonist and pirfenidone or a pirfenidone analog concurrently, with an amount effective to ameliorate the clinical course of the disease. The invention also features a method of treating alphavirus infection by administering to an individual interferon receptor agonist and pirfenidone or a pirfenidone analog in a synergistically effective amount to ameliorate the clinical course of the disease. The invention further features a method of treating alphaviral infection, generally involving administering to an individual interferon receptor agonist and pirfenidone or a pirfenidone analog concurrently, with an amount of the interferon receptor agonist that is at least about 90%, or at least about 95%, or at least about 100%, or at least about 110%, of the maximum tolerated dose (MTD) of the individual for the interferon receptor agonist if the same were to be used as a monotherapy for treatment of the alphaviral infection in the individual, in combination with an amount of pirfenidone or a pirfenidone analog effective to reduce the severity or incidence of side effects arising from such monotherapy, where the combination of the interferon receptor agonist and pirfenidone or a pirfenidone analog ameliorate the clinical course of the disease.

The invention features a method of treating West Nile viral infection, generally involving administering to an individual an interferon receptor agonist and pirfenidone or a pirfenidone analog concurrently, with an amount effective to reduce the time to viral clearance or to reduce morbidity or mortality in clinical outcomes. The invention also features a method of treating West Nile viral infection by administering to an individual an interferon receptor agonist and pirfenidone or a pirfenidone analog in a synergistically effective amount to reduce the time to viral clearance or to reduce morbidity or mortality in clinical outcomes. The invention further features a method of treating West Nile viral infection, generally involving administering to an individual interferon receptor agonist and pirfenidone or a pirfenidone analog concurrently, with an amount of the interferon receptor agonist that is at least about 90%, or at least about 95%, or at least about 100%, or at least about 110%, of the maximum tolerated dose (MTD) of the individual for interferon receptor agonist if the same were to be used as a monotherapy for treatment of the West Nile viral infection in the individual, in combination with an amount of pirfenidone or a pirfenidone analog effective to reduce the severity or incidence of side effects arising from such monotherapy, where the combination of the interferon receptor agonist and pirfenidone or a pirfenidone analog ameliorate the clinical course of the disease.

The invention features a method of treating hepatitis C virus (HCV) infection, generally involving administering to an individual an interferon receptor agonist and pirfenidone or a pirfenidone analog concurrently, with an amount effective to achieve a sustained viral response. The invention also features a method of treating HCV infection by administering to an individual an interferon receptor agonist and pirfenidone or a pirfenidone analog in a synergistically effective amount to achieve a sustained viral response. The invention further features a method of treating hepatitis C virus (HCV) infection, generally involving administering to an individual an interferon receptor agonist and pirfenidone or a pirfenidone analog concurrently, with an amount of the interferon receptor agonist that is at least about 90%, or at least about 95%, or at least about 100%, or at least about 110%, of the maximum tolerated dose (MTD) of the individual for the interferon receptor agonist if the same were to be used as a monotherapy for treatment of the HCV infection in the individual, in combination with an amount of pirfenidone or a pirfenidone analog effective to reduce the severity or incidence of side effects arising from such monotherapy, where the combination of the interferon receptor agonist and pirfenidone or a pirfenidone analog are effective to achieve a sustained viral response.

The invention features a method of reducing liver fibrosis in an individual, generally involving administering an interferon receptor agonist and pirfenidone or a pirfenidone analog concurrently, with an amount effective to reduce liver fibrosis. The invention also features a method of reducing liver fibrosis in an individual by administering an interferon receptor agonist and pirfenidone or a pirfenidone analog in a synergistically effective amount to reduce liver fibrosis. In some embodiments, the degree of liver fibrosis is determined by pre-treatment and post-treatment staging of a liver biopsy, wherein the stage of liver fibrosis, as measured by a standardized scoring system, is reduced by at least one unit when comparing pre-treatment with post-treatment liver biopsies.

The invention features a method of increasing liver function in an individual suffering from liver fibrosis, generally involving administering an interferon receptor agonist and pirfenidone or a pirfenidone analog concurrently, with an amount effective to increase a liver function. The invention also features a method of increasing liver function in an individual suffering from liver fibrosis by administering an interferon receptor agonist and pirfenidone or a pirfenidone analog in a synergistically effective amount to increase a liver function. Liver function may be indicated by measuring a parameter selected from the group consisting of serum transaminase level, prothrombin time, serum bilirubin level, blood platelet count, serum albumin level, improvement in portal wedge pressure, reduction in degree of ascites, reduction in a level of encephalopathy, and reduction in a degree of internal varices.

The invention features a method of reducing the incidence of a complication of cirrhosis of the liver. The methods generally involve administering an interferon receptor agonist and pirfenidone or a pirfenidone analog concurrently, with an amount effective to reduce the incidence of a complication of cirrhosis of the liver. The invention also features a method of reducing the incidence of a complication of cirrhosis of the liver by administering an interferon receptor agonist and pirfenidone or a pirfenidone analog in a synergistically effective amount to reduce the incidence of a complication of cirrhosis of the liver. Examples of complications of cirrhosis of the liver are portal hypertension, progressive liver insufficiency, and hepatocellular carcinoma.

In carrying out the methods of combination therapy for alphaviral infection, hepatitis C viral infection, West Nile viral infection and/or liver fibrosis in an individual as described above, an interferon receptor agonist and pirfenidone or a pirfenidone analog are administered to the individual. In some embodiments, the interferon receptor agonist and pirfenidone or a pirfenidone analog are administered in the same formulation. In other embodiments, the interferon receptor agonist and pirfenidone or a pirfenidone analog are administered in separate formulations. When administered in separate formulations, the interferon receptor agonist and pirfenidone or a pirfenidone analog can be administered substantially simultaneously, or can be administered within about 24 hours of one another. In many embodiments, the interferon receptor agonist is administered subcutaneously and pirfenidone or a pirfenidone analog is administered orally in multiple doses. Optionally, the interferon receptor agonist is administered to the individual by a controlled drug delivery device. Optionally, the interferon receptor agonist is administered to the individual substantially continuously or continuously by a controlled drug delivery device. Optionally, the controlled drug delivery device is an implantable infusion pump and the infusion pump delivers the interferon receptor agonist to the individual by subcutaneous infusion.

In some embodiments, the invention provides any one of the above-described methods in which the interferon receptor agonist is a Type I interferon receptor agonist. In other embodiments, the invention provides any one of the above-described methods in which the interferon receptor agonist is a Type II interferon receptor agonist. In other embodiments, the invention provides any one of the above-described methods in which the interferon receptor agonist is a Type III interferon receptor agonist.

In another aspect, the invention provides any of the above-described methods in which the interferon receptor agonist is an IFN-α. In some of these embodiments, the IFN-α is a consensus interferon. Optionally, the consensus interferon is INFERGEN® interferon alfacon-1.

In another aspect, the invention provides any of the above-described methods in which the interferon receptor agonist is IFN-α2a or IFN-α2b.

In another aspect, the invention provides any of the above-described methods in which the interferon receptor agonist is a PEGylated IFN-α. In some of these embodiments, the PEGylated IFN-α is PEGylated consensus IFN-α (CIFN). In some of these embodiments, the PEGylated IFN-α is PEGASYS® PEGylated IFN-α2a In some of these embodiments, the PEGylated IFN-α is PEG-INTRON® PEGylated IFN-α2b.

In other aspects, the invention provides any one of the above-described methods in which the interferon receptor agonist is an IFN-β.

In other aspects, the invention provides any one of the above-described methods in which the interferon receptor agonist is IFN-tau.

In other aspects, the invention provides any one of the above-described methods in which the interferon receptor agonist is IFN-ω.

In other aspects, the invention provides any one of the above-described methods in which the interferon receptor agonist is an IFN-γ.

In some embodiments, IFN-γ is co-administered with IFN-α and pirfenidone or a pirfenidone analog. In other embodiments, ribavirin is co-administered with an interferon receptor agonist and pirfenidone or a pirfenidone analog. In still other embodiments, ribavirin is co-administered with IFN-α, IFN-γ and pirfenidone (or a pirfenidone analog).

### BRIEF DESCRIPTION OF THE DRAWING

FIG. 1 depicts the amino acid sequence of the consensus interferon IFN-alpha con1 (SEQ ID NO:1).

FIG. 2 presents one way analysis of 19 ng interferon in combination with pirfenidone in a viral inhibition assay.

FIG. 3 presents one way analysis of 4.8 ng interferon in combination with pirfenidone in a viral inhibition assay.

FIG. 4 presents one way analysis of 1.2 ng interferon in combination with pirfenidone in a viral inhibition assay.

FIG. 5 presents one way analysis of 0.3 ng interferon in combination with pirfenidone in a viral inhibition assay.

FIG. 6 presents one way analysis of 0.076 ng interferon in combination with pirfenidone in a viral inhibition assay.

FIG. 7 presents one way analysis of 0.019 ng interferon in combination with pirfenidone in a viral inhibition assay.

FIG. 8 presents one way analysis of 0.0049 ng interferon in combination with pirfenidone in a viral inhibition assay.

FIG. 9 presents one way analysis of 0.001 ng interferon in combination with pirfenidone in a viral inhibition assay.

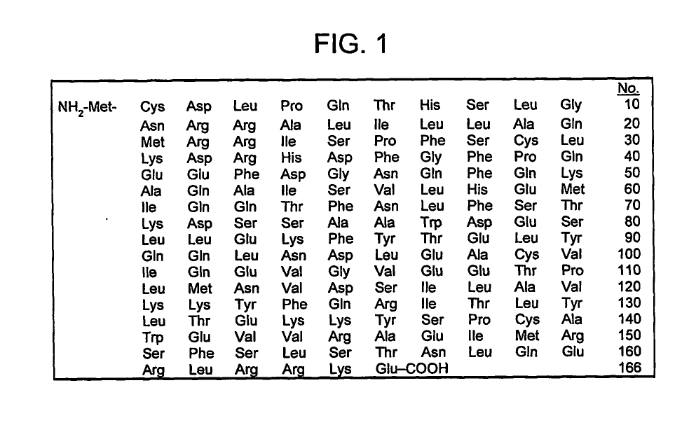

### DEFINIONS

As used herein, the term “hepatic fibrosis,” used interchangeably herein with “liver fibrosis,” refers to the growth of scar tissue in the liver that can occur in the context of a chronic hepatitis infection.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, equines, ungulates, and primates, including simians and humans.

As used herein, the term “interferon receptor agonist” refers to any agent that binds to an interferon receptor, which binding results in signal transduction via the receptor. Interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; chemical agonists; and the like.

As used herein, the term “alphavirus,” and its grammatical variants, refers to a group of viruses characterized by (i) an RNA genome (ii) viral replication in the cytoplasm of host cells and (iii) no DNA phase occurs in the viral replication cycle.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

The term “therapeutically effective amount” is meant an amount of a therapeutic agent, or a rate of delivery of a therapeutic agent, effective to facilitate a desired therapeutic effect The precise desired therapeutic effect will vary according to the condition to be treated, the formulation to be administered, and a variety of other factors that are appreciated by those of ordinary skill in the art.

The term “sustained viral response” (SVR; also referred to as a “sustained response” or a “durable response”), as used herein, refers to the response of an individual to a treatment regimen for HCV infection, in terms of serum HCV titer. Generally, a “sustained viral response” refers to no detectable HCV RNA (e.g., less than about 500, less than about 200, or less than about 100 genome copies per milliliter serum) found in the patient's serum for a period of at least about one month, at least about two months, at least about three months, at least about four months, at least about five months, or at least about six months following cessation of treatment.

The term “Units” refers to units of measurement for quantitation of the ability of the interferon to inhibit the cytopathic effect of a suitable virus (e.g. encephalomyocarditis virus (EMC), vesicular stomatitis virus, Semliki forest virus) after infection of an appropriate cell line (e.g., the human lung carcinoma cell lines, A549; HEP2/C; and the like). The antiviral activity is normalized to “Units” of antiviral activity exhibited by a reference standard such as human interferon alpha supplied by the World Health Organization. Such methods are detailed in numerous references. A particular method for measuring Units is described in Familletti, P. C., Rubinstein, S and Pestka, S. (1981) “A convenient and rapid cytopathic effect inhibition assay for interferon”, Methods in Enzymol, Vol 78 (S. Pestka, ed), Academic Press, New York pages 387-394. For the most part, the reference standard is human interferon alpha supplied by the World Health Organization, and the method for measuring International Units is that described in Familletti, supra.

The amounts of interferon receptor agonist administered will depend upon the specific activities of the particular interferon receptor agonist, and its biological performance in vivo. Thus, for example, the amounts-of interferon-alpha administered will depend on the specific activities of the IFN-α polypeptide and its biological performance in vivo. For example, IFN-α 2b is administered at 11.54 μg protein three times a week corresponding to 3×10⁶ U per injection (specific activity, 2.68×10⁶ IU/mg). On the other hand, CIFN alfa-con 1 is administered at 9 μg doses per injection corresponding to 9×10⁶ U per administration (specific activity, 1×10⁹ U/mg). However, in view of the fact that PEGylation reactions often result in a reduction in activity, larger mass doses of PEGylated material are administered to achieve efficacy (e.g. reduction in viral load; sustained viral response, etc.).

“Treatment failure patients” as used herein generally refers to HCV-infected patients who failed to respond to previous therapy for HCV (referred to as “non-responders”) or who initially responded to previous therapy, but in whom the therapeutic response was not maintained (referred to as “relapsers”). The previous therapy generally can include treatment with IFN-α monotherapy or IFN-α combination therapy, where the combination therapy may include administration of IFN-α and an antiviral agent such as ribavirin.

As used herein, the term “Type I interferon receptor agonist” refers to any naturally occurring or non-naturally occurring ligand of human Type I interferon receptor, which binds to and causes signal transduction via the receptor. Type I interferon receptor agonists include interferons, including naturally-occurring interferons, modified interferons, synthetic interferons, pegylated interferons, fusion proteins comprising an interferon and a heterologous protein, shuffled interferons; antibody agonists specific for an interferon receptor; non-peptide chemical agonists; and the like.

As used herein, the term “Type II interferon receptor agonist” refers to any naturally occurring or non-naturally occurring ligand of human Type II interferon receptor that binds to and causes signal transduction via the receptor. Type II interferon receptor agonists include native human interferon-γ, recombinant IFN-γ species, glycosylated IFN-γ species, pegylated IFN-γ species, modified or variant IFN-γ species, IFN-γ fusion proteins, antibody agonists specific for the receptor, non-peptide agonists, and the like.

As used herein, the term “Type III interferon receptor agonist” refers to any naturally occurring or non-naturally occurring ligand of humanIL-28 receptor a (“IL-28R”; the amino acid sequence of which is described by Sheppard, et al., infra.) that binds to and causes signal transduction via the receptor.

A “specific pirfenidone analog,” and all grammatical variants thereof, refers to, and is limited to, each and every pirfenidone analog shown in Table 1.

The term “pharmacokinetic profile,” as used herein, refers to the profile of the curve that results from plotting serum concentration of interferon receptor agonist (e.g., IFN-α) over time, following administration of the interferon receptor agonist to a subject. “Area under the curve,” or “AUC,” refers to the integrated area under the curve generated by plotting serum concentration of the interferon receptor agonist over time following administration of the interferon receptor agonist.

The term “hepatitis virus infection” refers to infection with one or more of hepatitis A, B, C, D, or E virus, with blood-borne hepatitis viral infection being of particular interest, particularly hepatitis C virus infection.

The term “dosing event” as used herein refers to administration of an antiviral agent to a patient in need thereof, which event may encompass one or more releases of an antiviral agent from a drug dispensing device. Thus, the term “dosing event,” as used herein, includes, but is not limited to, installation of a continuous delivery device (e.g., a pump or other controlled release injectible system); and a single subcutaneous injection followed by installation of a continuous delivery system.

“Continuous delivery” as used herein (e.g., in the context of “continuous delivery of a substance to a tissue”) is meant to refer to movement of drug to a delivery site, e.g., into a tissue in a fashion that provides for delivery of a desired amount of substance into the tissue over a selected period of time, where about the same quantity of drug is received by the patient each minute during the selected period of time.

“Controlled release” as used herein (e.g., in the context of “controlled drug release”) is meant to encompass release of substance (e.g., interferon receptor agonist, such as IFN-α) at a selected or otherwise controllable rate, interval, and/or amount, which is not substantially influenced by the environment of use. “Controlled release” thus encompasses, but is not necessarily limited to, substantially continuous delivery, and patterned delivery (e.g., intermittent delivery over a period of time that is interrupted by regular or irregular time intervals).

“Patterned” or “temporal” as used in the context of drug delivery is meant delivery of drug in a pattern, generally a substantially regular pattern, over a pre-selected period of time (e.g., other than a period associated with, for example a bolus injection). “Patterned” or “temporal” drug delivery is meant to encompass delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

The term “controlled drug delivery device” is meant to encompass any device wherein the release (e.g., rate, timing of release) of a drug or other desired substance contained therein is controlled by or determined by the device itself and not substantially influenced by the environment of use, or releasing at a rate that is reproducible within the environment of use.

By “substantially continuous” as used in, for example, the context of “substantially continuous infusion” or “substantially continuous delivery” is meant to refer to delivery of drug in a manner that is substantially uninterrupted for a pre-selected period of drug delivery, where the quantity of drug received by the patient during any 8 hour interval in the pre-selected period never falls to zero. Furthermore, “substantially continuous” drug delivery can also encompass delivery of drug at a substantially constant, pre-selected rate or range of rates (e.g., amount of drug per unit time, or volume of drug formulation for a unit time) that is substantially uninterrupted for a pre-selected period of drug delivery.

By “substantially steady state” as used in the context of a biological parameter that may vary as a function of time, it is meant that the biological parameter exhibits a substantially constant value over a time course, such that the area under the curve defined by the value of the biological parameter as a function of time for any 8 hour period during the time course (AUC₈ₕᵣ) is no more than about 20% above or about 20% below, and preferably no more than about 15% above or about 15% below, and more preferably no more than about 10% above or about 10% below, the average area under the curve of the biological parameter over an 8 hour period during the time course (AUC_{8hr average}). The AUC_{8hr average} is defined as the quotient (q) of the area under the curve of the biological parameter over the entirety of the time course (AUCₜₒₜₐₗ) divided by the number of 8 hour intervals in the time course (t_{total/1/3days}), i.e., q=(AUCₜₒₜₐₗ)/(t_{total1/3days}). For example, in the context of a serum concentration of a drug, the serum concentration of the drug is maintained at a substantially steady state during a time course when the area under the curve of serum concentration of the drug over time for any 8 hour period during the time course (AUC₈ₕᵣ) is no more than about 20% above or about 20% below the average area under the curve of serum concentration of the drug over an 8 hour period in the time course (AUC_{8hr average}),) i.e., the AUC₈ₕᵣ is no more than 20% above or 20% below the AUC_{8hr average} for the serum concentration of the drug over the time course.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a dosing event” includes a plurality of such events and reference to “the alphavirus” includes reference to one or more alphaviruses and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides methods for treating an alphavirus infection, including methods of treating West Nile viral infection and methods of treating HCV infection, and methods of treating liver fibrosis, including reducing clinical liver fibrosis, reducing the likelihood that liver fibrosis will occur, and reducing a parameter associated with liver fibrosis. The methods generally involve involving administering effective amounts of an interferon receptor agonist and pirfenidone (or a pirfenidone analog) in combination therapy. Of particular interest in many embodiments is treatment of humans.

The invention is based on the observation that low doses of pirfenidone, when administered in combination therapy with IFN-α, have a synergistic effect on reducing viral growth. It was observed that lower amounts of IFN-α, when administered in combination therapy with pirfenidone, are effective in treating a hepatitis C virus infection, compared to the amount of IFN-α required for IFN-α monotherapy. It was further observed that the side effects frequently observed with IFN-α monotherapy are reduced with IFN-α/pirfenidone combination therapy.

Thus, interferon receptor agonist/pirfenidone combination therapy confers a number of advantages over conventional IFN-α monotherapy. First, the effective amount of interferon receptor agonist, such as IFN-α, is lower than with IFN-α monotherapy. Secondly, where the interferon receptor agonist is an IFN-α, undesirable side effects of IFN-α are reduced. The reduction in IFN-α-induced side effects may be due in part to the reduced amount of IFN-α administered, and in part to the reduction in the occurrence or severity of IFN-α-induced side effects in response to pirfenidone therapy. A reduction in undesirable side effects of IFN-α decreases patient discomfort and increases patient compliance. Finally, IFN-α and pirfenidone, when administered in combination therapy, exhibit synergistic effects.

In some embodiments, the methods of the invention generally involve administering a therapeutically effective amount of an interferon receptor agonist and pirfenidone (or a pirfenidone analog) for the treatment of an alphavirus infection. In these embodiments, a “therapeutically effective amount” of an interferon receptor agonist and pirfenidone (or a pirfenidone analog) is an amount of interferon receptor agonist and pirfenidone (or a pirfenidone analog) that is effective in treating an alphavirus infection.

In some embodiments, the methods of the invention generally involve administering a therapeutically effective amount of an interferon receptor agonist and pirfenidone (or a pirfenidone analog) for the treatment of an HCV infection. In these embodiments, a “therapeutically effective amount” of an interferon receptor agonist and pirfenidone (or a pirfenidone analog) is an amount of interferon receptor agonist and pirfenidone (or a pirfenidone analog) that is effective in treating an HCV infection.

In many instances, HCV infection is associated with, or results in liver fibrosis. Thus, in some embodiments, the methods of the invention generally involve administering a therapeutically effective amount of IFN-α and pirfenidone (or a pirfenidone analog) for the treatment of liver fibrosis due to HCV infection. In these embodiments, a “therapeutically effective amount” of an interferon receptor agonist and pirfenidone (or a pirfenidone analog) is an amount of interferon receptor agonist and pirfenidone (or a pirfenidone analog) that is effective in treating liver fibrosis due to an HCV infection.

Liver fibrosis is a precursor to the complications associated with liver cirrhosis, such as portal hypertension, progressive liver insufficiency, and hepatocellular carcinoma. A reduction in liver fibrosis thus reduces the incidence of such future complications. Accordingly, the present invention further provides methods of reducing the likelihood that an individual will develop complications associated with cirrhosis of the liver.

In other embodiments, methods of the invention generally involve administering a therapeutically effective amount of an interferon receptor agonist and pirfenidone (or a pirfenidone analog) for the treatment of West Nile viral infection. In these embodiments, a “therapeutically effective amount” of an interferon receptor agonist and pirfenidone (or a pirfenidone analog) is an amount of interferon receptor agonist and pirfenidone (or a pirfenidone analog) that is effective in treating a West Nile viral infection.

Treatment Methods

The present invention provides methods for treating an alphavirus infection, and methods of treating liver fibrosis, involving administering effective amounts of an interferon receptor agonist and pirfenidone or a pirfenidone analog in combination therapy.

The methods and compositions described herein are generally useful in treatment of any alphavirus. Treatment of HCV infection is of particular interest in some embodiments. Reference to HCV herein is for illustration only and is not meant to be limiting.

Whether a subject method is effective in treating an alphaviral infection can be determined by a reduction in number or length of hospital stays, a reduction in time to viral clearance, a reduction of morbidity or mortality in clinical outcomes, a reduction in viral burden, or other indicator of disease response in the patient.

In general, an effective amount of an interferon receptor agonist and pirfenidone (or a pirfenidone analog) is an amount that is effective to reduce the time to viral clearance, or an amount that is effective to reduce morbidity or mortality in the clinical course of the disease, or an amount that is effective to improve some other indicator of disease response (e.g., an amount that is effective to reduce viral load; achieve a sustained viral response; etc.).

Whether a subject method is effective in treating an HCV infection can be determined by measuring viral load, or by measuring a parameter associated with HCV infection, including, but not limited to, liver fibrosis, elevations in serum transaminase levels, and necroinflammatory activity in the liver. Indicators of liver fibrosis are discussed in detail below.

The method involves administering an effective amount of an interferon receptor agonist in combination with an effective amount of pirfenidone or a pirfenidone analog. In some embodiments, effective amounts of an interferon receptor agonist and pirfenidone (or a pirfenidone analog) are amounts that are effective to reduce viral titers to undetectable levels, e.g., to about 1000 to about 5000, to about 500 to about 1000, or to about 100 to about 500 genome copies/mL serum. In some embodiments, effective amounts of an interferon receptor agonist and pirfenidone (or a pirfenidone analog) are amounts that are effective to reduce viral load to lower than 100 genome copies/mL serum.

In some embodiments, effective amounts of an interferon receptor agonist and pirfenidone (or a pirfenidone analog) are amounts that are effective to achieve a 1.5-log, a 2-log, a 2.5-log, a 3-log, a 3.5-log, a 4-log, a 4.5-log, or a 5-log reduction in viral titer in the serum of the individual.

In many embodiments, effective amounts of an interferon receptor agonist and pirfenidone (or a pirfenidone analog) are amounts that are effective to achieve a sustained viral response, e.g., no detectable HCV RNA (e.g., less than about 500, less than about 400, less than about 200, or less than about 100 genome copies per milliliter serum) is found in the patient's serum for a period of at least about one month, at least about two months, at least about three months, at least about four months, at least about five months, or at least about six months following cessation of therapy.

As noted above, whether a subject method is effective in treating an HCV infection can be determined by measuring a parameter associated with HCV infection, such as liver fibrosis. Methods of determining the extent of liver fibrosis are discussed in detail below. In some embodiments, the level of a serum marker of liver fibrosis indicates the degree of liver fibrosis.

As one non-limiting example, levels of serum alanine aminotransferase (ALT) are measured, using standard assays. In general, an ALT level of less than about 45 international units is considered normal. In some embodiments, an effective amount of an interferon receptor agonist and pirfenidone (or a pirfenidone analog) is an amount effective to reduce ALT levels to less than about 45 IU/ml serum.

A therapeutically effective amount of an interferon receptor agonist and pirfenidone (or a pirfenidone analog) is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Methods of measuring serum markers include immunological-based methods, e.g., enzyme-linked immunosorbent assays (ELISA), radioimmunoassays, and the like, using antibody specific for a given serum marker.

In many embodiments, the effective amounts of interferon receptor agonist and pirfenidone (or a pirfenidone analog) are synergistic amounts. As used herein, a “synergistic combination” or a “synergistic amount” of interferon receptor agonist and pirfenidone or a pirfenidone analog is a combined dosage that is more effective in the therapeutic or prophylactic treatment of a alphaviras infection than the incremental improvement in treatment outcome that could be predicted or expected from a merely additive combination of (i) the therapeutic or prophylactic benefit of interferon receptor agonist when administered at that same dosage as a monotherapy and (ii) the therapeutic or prophylactic benefit of pirfenidone or a pirfenidone analog when administered at the same dosage as a monotherapy.

In some embodiments of the invention, a selected amount of an interferon receptor agonist and a selected amount of pirfenidone or a pirfenidone analog are effective when used in combination therapy for a disease, but the selected amount of interferon receptor agonist and/or the selected amount of pirfenidone or a pirfenidone analog is ineffective when used in monotherapy for the disease. Thus, the invention encompasses (1) regimens in which a selected amount of pirfenidone or a pirfenidone analog enhances the therapeutic benefit of a selected amount of interferon receptor agonist when used in combination therapy for a disease, where the selected amount of pirfenidone or a pirfenidone analog provides no therapeutic benefit when used in monotherapy for the disease (2) regimens in which a selected amount of interferon receptor agonist enhances the therapeutic benefit of a selected amount of pirfenidone or a pirfenidone analog when used in combination therapy for a disease, where the selected amount of interferon receptor agonist provides no therapeutic benefit when used in monotherapy for the disease and (3) regimens in which a selected amount of interferon receptor agonist and a selected amount of pirfenidone or a pirfenidone analog provide a therapeutic benefit when used in combination therapy for a disease, where each of the selected amounts of interferon receptor agonist and pirfenidone or a pirfenidone analog, respectively, provides no therapeutic benefit when used in monotherapy for the disease. As used herein, a “synergistically effective amount” of interferon receptor agonist and pirfenidone or a pirfenidone analog, and its grammatical equivalents, shall be understood to include any regimen encompassed by any of (1)-(3) above.

In some embodiments, administration of effective amounts of interferon receptor agonist and pirfenidone or pirfenidone analog according to the invention reduces side effects frequently experienced by individuals treated with IFN-α and not pirfenidone or pirfenidone analog, e.g., IFN-α monotherapy. Side effects include, but are not limited to, fever, malaise, tachycardia, chills, headache, arthralgia, myalgia, myelosuppression, suicide ideation, platelet suppression, and anorexia. Side effects are reduced by at least about 10%, at least about 20%, at least about 30%, at least about 40%, at least about 50%, at least about 60%, or more, compared to the rate of occurrence or the degree or extent of the side effect when the interferon receptor agonist alone is administered. For example, if a fever is experienced with IFN-α monotherapy, then the body temperature of an individual treated with IFN-α/pirfenidone (or a pirfenidone analog) combination therapy according to the instant invention is reduced by at least 0.5 degree Fahrenheit, and in some embodiments is within the normal range, e.g., at or near 98.6° F.

West Nile Virus

The present invention provides methods for treating West Nile viral infection. The methods generally involve administering an interferon receptor agonist and pirfenidone (or a pirfenidone analog) to an individual in an amount that is effective to reduce the time to viral clearance in the individual, and/or to ameliorate the clinical course of the disease.

Whether a subject method is effective in treating a West Nile viral infections can be determined by a reduction in number or length of hospital stays, a reduction in time to viral clearance, a reduction of morbidity or mortality in clinical outcomes, or other indicator of disease response.

In general, effective amounts of interferon receptor agonist and pirfenidone (or a pirfenidone analog) are amounts that are effective to reduce the time to viral clearance, or an amount that is effective to reduce morbidity or mortality in the clinical course of the disease.

Effective amounts of interferon receptor agonist and pirfenidone (or a pirfenidone analog), as well as dosing regimens, are as discussed below.

Fibrosis

The instant-invention provides methods for treating liver fibrosis (including forms of liver fibrosis resulting from, or associated with, HCV infection), generally involving administering therapeutic amounts of an interferon receptor agonist and pirfenidone (or a pirfenidone analog). Effective amounts of interferon receptor agonist and pirfenidone (or a pirfenidone analog), as well as dosing regimens, are as discussed below.

Whether treatment with an interferon receptor agonist and pirfenidone (or a pirfenidone analog) is effective in reducing liver fibrosis is determined by any of a number of well-established techniques for measuring liver fibrosis and liver function. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite. The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, a therapeutically effective amount of an interferon receptor agonist and pirfenidone (or a pirfenidone analog) is an amount of an interferon receptor agonist and pirfenidone (or a pirfenidone analog) that effects a change of one unit or more in the fibrosis stage based on pre- and post-therapy liver biopsies. In particular embodiments, a therapeutically effective amount of an interferon receptor agonist and pirfenidone (or a pirfenidone analog) reduces liver fibrosis by at least one unit in the METAVIR, the Knodell, the Scheuer, the Ludwig, or the Ishak scoring system.

Secondary, or indirect, indices of liver function can also be used to evaluate the efficacy of IFN-α and pirfenidone (or a pirfenidone analog) treatment. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score.

An effective amount of an interferon receptor agonist and pirfenidone (or a pirfenidone analog) is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or to a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

A therapeutically effective amount of an interferon receptor agonist and pirfenidone (or a pirfenidone analog) is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

Quantitative tests of functional liver reserve can also be used to assess the efficacy of treatment with an interferon receptor agonist and pirfenidone (or a pirfenidone analog). These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

A therapeutically effective amount of an interferon receptor agonist and pirfenidone (or a pirfenidone analog) is an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or to a placebo-treated individual.

Whether treatment with an interferon receptor agonist and pirfenidone (or a pirfenidone analog) is effective in reducing the incidence of a disorder associated with cirrhosis of the liver can readily be determined by those skilled in the art.

Reduction in liver fibrosis increases liver function. Thus, the invention provides methods for increasing liver function, generally involving administering a therapeutically effective amount of an interferon receptor agonist and pirfenidone (or a pirfenidone analog). Liver functions include, but are not limited to, synthesis of proteins such as serum proteins (e.g., albumin, clotting factors, alkaline phosphatase, aminotransferases (e.g., alanine transarninase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal level of alanine transaminase is about 45 IU per milliliter of serum. The normal range of aspartate transaminase is from about 5 to about 40 units per liter of serum. Bilinibin is measured using standard assays. Normal bilinibin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

A therapeutically effective amount of an interferon receptor agonist and pirfenidone (or a pirfenidone analog) is one that is effective to increase liver function by at least about 10%, at least about 20%, at least about 30%, at least about 40%, at least about 50%, at least about 60%, at least about 70%, at least about 80%, or more. For example, a therapeutically effective amount of an interferon receptor agonist and pirfenidone (or a pirfenidone analog) is an amount effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of an interferon receptor agonist and pirfenidone (or a pirfenidone analog) is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

Pirfenidone and Analogs Thereof

Pirfenidone (5-methyl-1-phenyl-2-(1H)-pyridone) and specific pirfenidone analogs are disclosed for the treatment of fibrotic conditions. A “fibrotic condition” is one that is amenable to treatment by administration of a compound having anti-fibrotic activity. 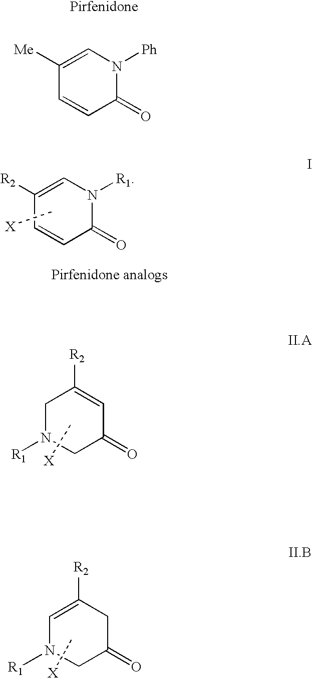
Descriptions for Substituents R₁, R₂, X

R₁: carbocyclic (saturated and unsaturated), heterocyclic (saturated or unsaturated), allcyls (saturated and unsaturated). Examples include phenyl, benzyl, pyrimidyl, naphthyl, indolyl, pyrrolyl, furyl, thienyl, imidazolyl, cyclohexyl, piperidyl, pyrrolidyl, morpholinyl, cyclohexenyl, butadienyl, and the like.

R₁ can further include substitutions on the carbocyclic or heterocyclic moieties with substituents such as halogen, nitro, amino, hydroxyl, alkoxy, carboxyl, cyano, thio, alkyl, aryl, heteroalkyl, heteroaryl and combinations thereof, for example, 4-nitrophenyl, 3-chlorophenyl, 2,5-dinitrophenyl, 4-methoxyphenyl, 5-methyl-pyrrolyl, 2,5-dichlorocyclohexyl, guanidinyl-cyclohexenyl and the like.

R₂: alkyl, carbocylic, aryl, heterocyclic. Examples include: methyl, ethyl, propyl, isopropyl, phenyl, 4-nitrophenyl, thienyl and the like.

X: may be any number (from 1 to 3) of substituents on the carbocyclic or heterocyclic ring. The substituents can be the same or different. Substituents can include hydrogen, alkyl, heteroalkyl, aryl, heteroaryl, halo, nitro, carboxyl, hydroxyl, cyano, amino, thio, alkylamino, haloaryl and the like.

The substituents may be optionally further substituted with 1-3 substituents from the group consisting of alkyl, aryl, nitro, alkoxy, hydroxyl and halo groups. Examples include: methyl, 2,3-dimethyl, phenyl, p-tolyl, 4-chlorophenyl, 4-nitrophenyl, 2,5-dichlorophenyl, furyl, thienyl and the like.

TABLE 1

| IA | IIB |
|---|---|
| 5-Methyl-1-(2′-pyridyl)-2- | 6-Methyl-1-phenyl- |
| (1H)pyridine, | 3-(1H)pyridone, |
| 6-Methyl-1-phenyl-2-(1H) pyridone, | 5-Methyl-1-p-tolyl- |
| | 3-(1H)pyridone, |
| 5-Methyl-3-phenyl-1-(2′-thienyl)- | 5-Methyl-1-(2′- |
| 2-(1H)pyridone, | naphthyl)-3- |
| | (1H)pyridone, |
| 5-Methyl-1-(2′-naphthyl)-2- | 5-Methyl-1-phenyl- |
| (1H)pyridone, | 3-(1H)pyridone, |
| 5-Methyl-1-p-tolyl-2-(1H)pyridone, | 5-Methyl-1-(5′- |
| | quinolyl)-3- |
| | (1H)pyridone, |
| 5-Methyl-1-(1′naphthyl)-2- | 5-Ethyl-1-phenyl- |
| (1H)pyridone, | 3-(1H)pyridone, |
| 5-Ethyl-1-phenyl-2-(1H)pyridone, | 5-Methyl-1-(4′- |
| | methoxyphenyl)-3- |
| | (1H)pyridone, |
| 5-Methyl-1-(5′-quinolyl)-2- | 4-Methyl-1-phenyl- |
| (1H)pyridone, | 3-(1H)pyridone, |
| 5-Methyl-1-(4′-quinolyl)-2- | 5-Methyl-1-(3′- |
| (1H)pyridone, | pyridyl)-3- |
| | (1H)pyridone, |
| 5-Methyl-1-(4′-pyridyl)-2- | 5-Methyl-1-(2′- |
| (1H)pyridone, | Thienyl)-3- |
| | (1H)pyridone, |
| 3-Methyl-1-phenyl-2-(1H)pyridone, | 5-Methyl-1-(2′- |
| | pyridyl)-3- |
| | (1H)pyridone, |
| 5-Methyl-1-(4′-methoxyphenyl)- | 5-Methyl-1-(2′- |
| 2-(1H)pyridone, | quinolyl)-3- |
| | (1H)pyridone, |
| 1-Phenyl-2-(1H)pyridone, | 1-Phenyl-3- |
| | (1H)pyridine, |
| 1,3-Diphenyl-2-(1H)pyridone, | 1-(2′-Furyl)- |
| | 5-methyl-3- |
| | (1H)pyridone, |
| 1,3-Diphenyl-5-methyl-2- | 1-(4′- |
| (1H)pyridone, | Chlorophenyl)- |
| | 5-methyl-3- |
| | (1H)pyridine. |
| 5-Methyl-1-(3′-trifluorometh- | |
| ylphenyl)-2-(1H)-pyridone, | |
| 3-Ethyl-1-phenyl-2-(1H)pyridone, | |
| 5-Methyl-1-(3′-pyridyl)-2- | |
| (1H)pyridone, | |
| 5-Methyl-1-(3-nitrophenyl)-2- | |
| (1H)pyridone, | |
| 3-(4′-Chlorophenyl)-5- | |
| Methyl-1-phenyl-2-(1H)pyridone, | |
| 5-Methyl-1-(2′-Thienyl)-2- | |
| (1H)pyridone, | |
| 5-Methyl-1-(2′-thiazolyl)- | |
| 2-(1H)pyridone, | |
| 3,6-Dimethyl-1-phenyl-2- | |
| (1H)pyridone, | |
| 1-(4′Chlorophenyl)-5- | |
| Methyl-2-(1H)pyridone, | |
| 1-(2′-Imidazolyl)-5- | |
| Methyl-2-(1H)pyridone, | |
| 1-(4′-Nitrophenyl)-2- | |
| (1H)pyridone, | |
| 1-(2′-Furyl)-5-Methyl- | |
| 2-(1H)pyridone, | |
| 1-Phenyl-3-(4′-chloro- | |
| phenyl)-2-(1H)pyridine. | |

U.S. Pat. Nos. 3,974,281; 3,839,346; 4,042,699; 4,052,509; 5,310,562; 5,518,729; 5,716,632; and 6,090,822 describe methods for the synthesis and formulation of pirfenidone and specific pirfenidone analogs in pharmaceutical compositions suitable for use in the methods of the present invention.

Agonists of Type I Interferon Receptors

In any of the above-described methods or apparatus, the interferon receptor agonist is in some embodiments an agonist of a Type I interferon receptor (e.g., “a Type I interferon agonist”). Type I interferon receptor agonists include an IFN-α; an IFN-β; an IFN-tau; an IFN-ω; antibody agonists specific for a Type I interferon receptor; and any other agonist of Type I interferon receptor, including non-polypeptide agonists.

IFN-α

The term “interferon-alpha” as used herein refers to a family of related polypeptides that inhibit viral replication and cellular proliferation and modulate immune response. The term “IFN-α” includes IFN-α polypeptides that are naturally occurring; non-naturally-occurring IFN-α polypeptides; and analogs of naturally occurring or non-naturally occurring IFN-α that retain antiviral activity of a parent naturally-occurring or non-naturally occurring IFN-α.

Suitable alpha interferons include, but are not limited to, naturally-occurring IFN-α (including, but not limited to, naturally occurring IFN-α2a, IFN-α2b); recombinant interferon alpha-2b such as Intron®A interferon available from Schering Corporation, Kenilworth, N.J.; recombinant interferon alpha-2a such as Roferon® interferon available from Hoffmann-La Roche, Nutley, N.J.; recombinant interferon alpha-2C such as Berofor® alpha 2 interferon available from Boehringer Ingelheim Pharmaceutical, Inc., Ridgefield, Conn.; interferon alpha-n1, a purified blend of natural alpha interferons such as Sumiferon available from Sumitomo, Japan or as Wellferon® interferon alpha-n1 (INS) available from the Glaxo-Wellcome Ltd., London, Great Britain; and interferon alpha-n3 a mixture of natural alpha interferons made by Interferon Sciences and available from the Purdue Frederick Co., Norwalk, Conn., under the Alferon® Tradename.

The term “IFN-α,” as used herein, also encompasses consensus IFN-α. As used herein, the term “consensus IFN-α” refers to a non-naturally-occurring polypeptide, which includes those amino acid residues that are common to all naturally-occurring human leukocyte IFN-α subtype sequences and which includes, at one or more of those positions where there is no amino acid common to all subtypes, an amino acid which predominantly occurs at that position, provided that at any such position where there is no amino acid common to all subtypes, the polypeptide excludes any amino acid residue which is not present in at least one naturally-occurring subtype. Amino acid residues that are common to all naturally-occurring human leukocyte IFN-α subtype sequences (“common amino acid residues”), and amino acid residues that occur predominantly at non-common residues (“consensus amino acid residues”) are known in the art.

The term “IFN-α” also encompasses consensus IFN-α. Consensus IFN-α (also referred to as “CIFN” and “IFN-con” and “consensus interferon”) encompasses but is not limited to the amino acid sequences designated IFN-con₁, IFN-con₂ and IFN-con₃ which are disclosed in U.S. Pat. Nos. 4,695,623 and 4,897,471; and consensus interferon as defined by determination of a consensus sequence of naturally occurring interferon alphas (e.g., Infergen®, InterMune, Inc., Brisbane, Calif.). IFN-con₁ is the consensus interferon agent in the Infergen® alfacon-1 product. The Infergen®consensusinterferon product is referred to herein by its brand name (Infergen®) or by its generic name (interferon alfacon-1). DNA sequences encoding IFN-con may be synthesized as described in the aforementioned patents or other standard methods. Use of CIFN is of particular interest

Also suitable for use in the present invention are fusion polypeptides comprising an IFN-α and a heterologous polypeptide. Suitable IFN-α fusion polypeptides include, but are not limited to, Albuferon-alpha™ (a fusion product of human albumin and IFN-α; Human Genome Sciences; see, e.g., Osborn et al. (2002) J. Pharmacol. Exp. Therap. 303:540-548). Also suitable for use in the present invention are gene-shuffled forms of IFN-α. See., e.g., Masci et al. (2003) Curr. Oncol. Rep. 5:108-113.

IFN-α polypeptides can be produced by any known method. DNA sequences encoding IFN-con may be synthesized as described in the above-mentioned patents or other standard methods. In many embodiments, IFN-α polypeptides are the products of expression of manufactured DNA sequences transformed or transfected into bacterial hosts, e.g., E. coli, or in eukaryotic host cells (e.g., yeast; mammalian cells, such as CHO cells; and the like). In these embodiments, the IFN-α is “recombinant IFN-α.” Where the host cell is a bacterial host cell, the IFN-α is modified to comprise an N-terminal methionine. IFN-α produced in E. coli is generally purified by procedures known to those skilled in the art and generally described in Klein et al. ((1988) J. Chromatog. 454:205-215) for IFN-con₁.

Bacterially produced IFN-α may comprise a mixture of isoforms with respect to the N-terminal amino acid residue. For example, purified IFN-con may comprise a mixture of isoforms with respect to the N-terminal methionine status. For example, in some embodiments, an IFN-con comprises a mixture of N-terminal methionyl IFN-con, des-methionyl IFN-con with an unblocked N-terminus, and des-methionyl IFN-con with a blocked N-terminus. As one non-limiting example, purified IFN-con, comprises a mixture of methionyl IFN-con₁ des-methionyl IFN-con₁ and des-methionyl IFN-con₁ with a blocked N-terminus. Klein et al. ((1990) Arch. Biochemistry & Biophys. 276:531-537). Alternatively, IFN-con may comprise a specific, isolated isoform. Isoforms of IFN-con are separated from each other by techniques such as isoelectric focusing which are known to those skilled in the art.

It is to be understood that IFN-α as described herein may comprise one or more modified amino acid residues, e.g., glycosylations, chemical modifications, and the like.

PEGylated IFN-α

The term “IFN-α” also encompasses derivatives of IFN-α that are derivatized (e.g., are chemically modified) to alter certain properties such as serum half-life. As such, the term “IFN-α” includes glycosylated IFN-α; IFN-α derivatized with polyethylene glycol (“PEGylated IFN-α”); and the like. PEGylated IFN-α, and methods for making same, is discussed in, e.g., U.S. Pat. Nos. 5,382,657; 5,981,709; and 5,951,974. PEGylated IFN-α encompasses conjugates of PEG and any of the above-described IFN-α molecules, including, but not limited to, PEG conjugated to interferon alpha-2a (Roferon, Hoffman La-Roche, Nutley, N.J.), interferon alpha 2b (Intron, Schering-Plough, Madison, N.J.), interferon alpha-2c (Berofor Alpha, Boehringer Ingelheim, Ingelheim, Germany); and consensus interferon as defined by determination of a consensus sequence of naturally occurring interferon alphas (Infergens, InterMune, Inc., Brisbane, Calif.).

Any of the above-mentioned IFN-α polypeptides can be modified with one or more polyethylene glycol moieties, i.e., PEGylated. The PEG molecule of a PEGylated IFN-α polypeptide is conjugated to one or more amino acid side chains of the IFN-α polypeptide. In some embodiments, the PEGylated IFN-α contains a PEG moiety on only one amino acid. In other embodiments, the PEGylated IFN-α contains a PEG moiety on two or more amino acids, e.g., the IFN-α contains a PEG moiety attached to two, three, four, five, six, seven, eight, nine, or ten different amino acid residues.

IFN-α may be coupled directly to PEG (i.e., without a linking group) through an amino group, a sulfhydryl group, a hydroxyl group, or a carboxyl group. In some embodiments, the PEGylated IFN-α is PEGylated at or near the amino terminus (N-terminus) of the IFN-α polypeptide, e.g., the PEG moiety is conjugated to the IFN-α polypeptide at one or more amino acid residues from amino acid 1 through amino acid 4, or from amino acid 5 through about 10.

In other embodiments, the PEGylated IFN-α is PEGylated at one or more amino acid residues from about 10 to about 28.

In other embodiments, the PEGylated IFN-α is PEGylated at or near the carboxyl terminus (C-terminus) of the IFN-α polypeptide, e.g., at one or more residues from amino acids 156-166, or from amino acids 150 to 155.

In other embodiments, the PEGylated IFN-α is PEGylated at one or more amino acid residues at one or more residues from amino acids 100-114.

Selection of the attachment site of polyethylene glycol on the IFN-α is determined by the role of each of the sites within the receptor-binding and/or active site domains of the protein, as would be known to the skilled artisan. In general, amino acids at which PEGylation is to be avoided include amino acid residues from amino acid 30 or amino acid 40; and amino acid residues from amino acid 113 to amino acid 149.

In some embodiments, PEG is attached to IFN-α via a linking group. The king group is any biocompatible linking group, where “biocompatible” indicates that the compound or group is non-toxic and may be utilized in vitro or in vivo without causing injury, sickness, disease, or death. PEG can be bonded to the linking group, for example, via an ether bond, an ester bond, a thiol bond or an amide bond. Suitable biocompatible linking groups include, but are not limited to, an ester group, an amide group, an imide group, a carbamate group, a carboxyl group, a hydroxyl group, a carbohydrate, a succinimide group (including, for example, succinimidyl succinate (SS), succinimidyl propionate (SPA), succinimidyl carboxymethylate (SCM), succinimidyl succinamide (SSA) or N-hydroxy succinimide (NHS)), an epoxide group, an oxycarbonylimidazole group (including, for example, carbonyldimidazole (CDI)), a nitro phenyl group (including, for example, nitrophenyl carbonate (NPC) or trichlorophenyl carbonate (TPC)), a trysylate group, an aldehyde group, an isocyanate group, a vinylsulfone group, a tyrosine group, a cysteine group, a histidine group or a primary amine. Methods for attaching a PEG to an IFN-α polypeptide are known in the art, and any known method can be used. See, for example, by Park et al, Anticancer Res., 1:373-376 (1981); Zaplipsky and Lee, Polyethylene Glycol Chemistry: Biotechnical and Biomedical Applications, J. M. Harris, ed., Plenum Press, NY, Chapter 21 (1992); and U.S. Pat. No. 5,985,265.

Pegylated IFN-α, and methods for making same, are discussed in, e.g., U.S. Pat. Nos. 5,382,657; 5,981,709; 5,985,265; and 5,951,974. Pegylated IFN-α encompasses conjugates of PEG and any of the above-described IFN-α molecules, including, but not limited to, PEG conjugated to interferon alpha-2a (Roferon, Hoffman LaRoche, Nutley, N.J.), where PEGylated Roferon is known as PEGASYS® (Hoffman LaRoche); interferon alpha 2b (Intron, Schering-Plough, Madison, N.J.), where PEGylated Intron is known as PEG-INTRON® (Schering-Plough); interferon alpha-2c (Berofor Alpha, Boehringer Ingelheim, Ingelheim, Germany); and consensus interferon (CIFN) as defined by determination of a consensus sequence of naturally occurring interferon alphas (Infergen, Amgen, Thousand Oaks, Calif.), where PEGylated Infergen is referred to as PEG-INFERGEN®.

In some embodiments, the PEGylated IFN-α comprises CIFN PEGylated at the epsilon amino group of a lysine residue.

Generally, the PEG moiety is linked to a surface-exposed lysine (“lys”) residue. Whether a lysine is surface exposed can be determined using any known method. Generally, analysis of hydrophilicity (e.g., Kyte-Doolittle and Hoppe-Woods analysis) and/or predicted surface-forming regions (e.g., Emini surface-forming probability analysis) is carried out using appropriate computer programs, which are well known to those skilled in the art. Suitable computer programs include PeptideStructure, and the like. Alternatively, NMR investigations can identify the surface accessible residues by virtue of the chemical shift of the protons of a specific functional group in the spectrum. In other cases, the inaccessibility or accessibility of residues to solvents or environment can be assessed by fluorescence. In yet other cases, the surface exposure of accessible lysines can be ascertained by the chemical reactivity to water soluble reagents e.g., Trinitrobenzene sulfonate or TNBS, and like measurements.

Polyethylene Glycol

Polyethylene glycol suitable for conjugation to an IFN-α polypeptide is soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons.

In many embodiments, PEG has at least one hydroxyl group, e.g., a terminal hydroxyl group, which hydroxyl group is modified to generate a functional group that is reactive with an amino group, e.g., an epsilon amino group of a lysine residue, a free amino group at the N-terminus of a polypeptide, or any other amino group such as an amino group of asparagine, glutamine, arginine, or histidine.

In other embodiments, PEG is derivatized so that it is reactive with free carboxyl groups in the IFN-α polypeptide, e.g., the free carboxyl group at the carboxyl terminus of the IFN-α polypeptide. Suitable derivatives of PEG that are reactive with the free carboxyl group at the carboxyl-terminus of IFN-α include, but are not limited to PEG-amine, and hydrazine derivatives of PEG (e.g., PEG-NH—NH₂).

In other embodiments, PEG is derivatized such that it comprises a terminal thiocarboxylic acid group, —COSH, which selectively reacts with amino groups to generate amide derivatives. Because of the reactive nature of the thio acid, selectivity of certain amino groups over others is achieved. For example, —SH exhibits sufficient leaving group ability in reaction with N-terminal amino group at appropriate pH conditions such that the ε-amino groups in lysine residues are protonated and remain non-nucleophilic. On the other hand, reactions under suitable pH conditions may make some of the accessible lysine residues to react with selectivity.

In other embodiments, the PEG comprises a reactive ester such as an N-hydroxy succinimidate at the end of the PEG chain. Such an N-hydroxysuccinimidate-containing PEG molecule reacts with select amino groups at particular pH conditions such as neutral 6.5-7.5. For example, the N-terminal amino groups may be selectively modified under neutral pH conditions. However, if the reactivity of the reagent were extreme, accessible-NH₂ groups of lysine may also react.

The PEG can be conjugated directly to the IFN-α polypeptide, or through a linker. In some embodiments, a linker is added to the IFN-α polypeptide, forming a linker-modified IFN-α polypeptide. Such linkers provide various functionalities, e.g., reactive groups such sulfhydryl, amino, or carboxyl groups to couple a PEG reagent to the linker-modified IFN-α polypeptide.

In some embodiments, the PEG conjugated to the IFN-α polypeptide is linear. In other embodiments, the PEG conjugated to the IFN-α polypeptide is branched. Branched PEG derivatives such as those described in U.S. Pat. No. 5,643,575, “star-PEG's” and multi-armed PEG's such as those described in Shearwater Polymers, Inc. catalog “Polyethylene Glycol Derivatives 1997-1998.” Star PEGs are described in the art including, e.g., in U.S. Pat. No. 6,046,305.

PEG having a molecular weight in a range of from about 2 kDa to about 100 kDa, is generally used, where the term “about,” in the context of PEG, indicates that in preparations of polyethylene glycol, some molecules will weigh more, some less, than the stated molecular weight. For example, PEG suitable for conjugation to IFN-α has a molecular weight of from about 2 kDa to about 5 kDa, from about 5 kDa to about 10 kDa, from about 10 kDa to about 15 kDa, from about 15 kDa to about 20 kDa, from about 20 kDa to about 25 kDa, from about 25 kDa to about 30 kDa, from about 30 kDa to about 40 kDa, from about 40 kDa to about 50 kDa, from about 50 kDa to about 60 kDa, from about 60 kDa to about 70 kDa, from about 70 kDa to about 80 kDa, from about 80 kDa to about 90 kDa, or from about 90 kDa to about 100 kDa.

Preparing PEG-IFN-α Conjugates

As discussed above, the PEG moiety can be attached, directly or via a linker, to an amino acid residue at or near the N-terminus, internally, or at or near the C-terminus of the IFN-α polypeptide. Conjugation can be carried out in solution or in the solid phase.

N-Terminal Linkage

Methods for attaching a PEG moiety to an amino acid residue at or near the N-terminus of an IFN-α polypeptide are known in the art. See, e.g., U.S. Pat. No. 5,985,265.

In some embodiments, known methods for selectively obtaining an N-terminally chemically modified IFN-α are used. For example, a method of protein modification by reductive alkylation which exploits differential reactivity of different types of primary amino groups (lysine versus the N-terminus) available for derivatization in a particular protein can be used. Under the appropriate reaction conditions, substantially selective derivatization of the protein at the N-terminus with a carbonyl group containing polymer is achieved. The reaction is performed at pH which allows one to take advantage of the pKₐ differences between the ε-amino groups of the lysine residues and that of the α-amino group of the N-terminal residue of the protein. By such selective derivatization attachment of a PEG moiety to the IFN-α is controlled: the conjugation with the polymer takes place predominantly at the N-terminus of the IFN-α and no significant modification of other reactive groups, such as the lysine side chain amino groups, occurs.

C-Terminal Linkage

N-terminal-specific coupling procedures such as described in U.S. Pat. No. 5,985,265 provide predominantly monoPEGylated products. However, the purification procedures aimed at removing the excess reagents and minor multiply PEGylated products remove the N-terminal blocked polypeptides. In terms of therapy, such processes lead to significant increases in manufacturing costs. For example, examination of the structure of the well-characterized Infergen® Alfacon-1 CIFN polypeptide amino acid sequence reveals that the clipping is approximate 5% at the carboxyl terminus and thus there is only one major C-terminal sequence. Thus, in some embodiments, N-terminally PEGylated IFN-α is not used; instead, the IFN-α polypeptide is C-terminally PEGylated.

An effective synthetic as well as therapeutic approach to obtain mono PEGylated Infergen product is therefore envisioned as follows:

A PEG reagent that is selective for the C-terminal can be prepared with or without spacers. For example, polyethylene glycol modified as methyl ether at one end and having an amino function at the other end may be used as the starting material.

Preparing or obtaining a water-soluble carbodiimide as the condensing agent can be carried out. Coupling IFN-α (e.g., Infergen® Alfacon-1 CIFN or consensus interferon) with a water-soluble carbodiimide as the condensing reagent is generally carried out in aqueous medium with a suitable buffer system at an optimal pH to effect the amide linkage. A high molecular weight PEG can be added to the protein covalently to increase the molecular weight.

The reagents selected will depend on process optimization studies. A non-limiting example of a suitable reagent is EDAC or 1-ethyl-3-(3-dimethylaminopropyl) carbodiimide. The water solubility of EDAC allows for direct addition to a reaction without the need for prior organic solvent dissolution. Excess reagent and the isourea formed as the by-product of the cross-linking reaction are both water-soluble and may easily be removed by dialysis or gel filtration. A concentrated solution of EDAC in water is prepared to facilitate the addition of a small molar amount to the reaction. The stock solution is prepared and used immediately in view of the water labile nature of the reagent. Most of the synthetic protocols in literature suggest the optimal reaction medium to be in pH range between 4.7 and 6.0. However the condensation reactions do proceed without significant losses in yields up to pH 7.5. Water may be used as solvent. In view of the contemplated use of Infergen, preferably the medium will be 2-(N-morpholino)ethane sulfonic acid buffer pre-titrated to pH between 4.7 and 6.0. However, 0.1M phosphate in the pH 7-7.5 may also be used in view of the fact that the product is in the same buffer. The ratios of PEG amine to the IFN-α molecule is optimized such that the C-terminal carboxyl residue(s) are selectively PEGylated to yield monoPEGylated derivative(s).

Even though the use of PEG amine has been mentioned above by name or structure, such derivatives are meant to be exemplary only, and other groups such as hydrazine derivatives as in PEG-NH—NH₂ which will also condense with the carboxyl group of the IFN-α protein, can also be used. In addition to aqueous phase, the reactions can also be conducted on solid phase. Polyethylene glycol can be selected from list of compounds of molecular weight ranging from 300-40000. The choice of the various polyethylene glycols will also be dictated by the coupling efficiency and the biological performance of the purified derivative in vitro and in vivo i.e., circulation times, anti viral activities etc.

Additionally, suitable spacers can be added to the C-terminal of the protein. The spacers may have reactive groups such as SH, NH₂ or COOH to couple with appropriate PEG reagent to provide the high molecular weight IFN-α derivatives. A combined solid/solution phase methodology can be devised for the preparation of C-terminal pegylated interferons. For example, the C-terminus of IFN-α is extended on a solid phase using a Gly-Gly-Cys-NH₂ spacer and then monopegylated in solution using activated dithiopyridyl-PEG reagent of appropriate molecular weights. Since the coupling at the C-terminus is independent of the blocking at the N-terminus, the envisioned processes and products will be beneficial with respect to cost (a third of the protein is not wasted as in N-terminal PEGylation methods) and contribute to the economy of the therapy to treat chronic hepatitis C infections, liver fibrosis etc.

There may be a more reactive carboxyl group of amino acid residues elsewhere in the molecule to react with the PEG reagent and lead to monoPEGylation at that site or lead to multiple PEGylations in addition to the —COOH group at the C-terminus of the IFN-α. It is envisioned that these reactions will be minimal at best owing to the steric freedom at the C-terminal end of the molecule and the steric hindrance imposed by the carbodiimides and the PEG reagents such as in branched chain molecules. It is therefore the preferred mode of PEG modification for Infergen and similar such proteins, native or expressed in a host system, which may have blocked N-termini to varying degrees to improve efficiencies and maintain higher in vivo biological activity.

Another method of achieving C-terminal PEGylation is as follows. Selectivity of C-terminal PEGylation is achieved with a sterically hindered reagent which excludes reactions at carboxyl residues either buried in the helices or internally in IFN-α. For example, one such reagent could be a branched chain PEG ˜40 kd in molecular weight and this agent could be synthesized as follows:

OH₃C—(CH₂CH₂O)n-CH₂CH₂NH₂+Glutamic Acid i.e., HOCO—CH₂CH₂CH(NH2)-COOH is condensed with a suitable agent e.g., dicyclohexyl carbodiimide or water-soluble EDC to provide the branched chain PEG agent OH₃C—(CH₂CH₂O)ₙ—CH₂CH₂NHCOCH(NH₂)CH₂OCH₃—(CH₂CH₂O)ₙ—CH₂CH₂NHCOCH₂.

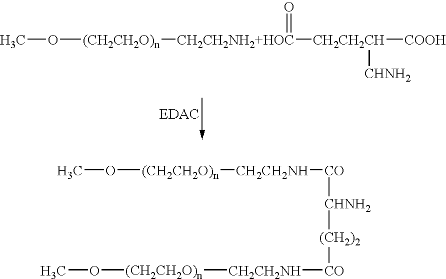

This reagent can be used in excess to couple the amino group with the free and flexible carboxyl group of IFN-α to form the peptide bond.

If desired, PEGylated IFN-α is separated from unPEGylated IFN-α using any known method, including, but not limited to, ion exchange chromatography, size exclusion chromatography, and combinations thereof. For example, where the PEG-IFN-α conjugate is a monoPEGylated IFN-α, the products are first separated by ion exchange chromatography to obtain material having a charge characteristic of monoPEGylated material (other multi-PEGylated material having the same apparent charge may be present), and then the monoPEGylated materials are separated using size exclusion chromatography.

Mixed Populations of IFN-α

In some embodiments, the IFN-α administered is a population of IFN-α polypeptides comprising PEGylated IFN-α polypeptides and non-PEGylated IFN-α polypeptides. Generally, a PEGylated IFN-α species represents from about 0.5% to about 99.5% of the total population of IFNα polypeptide molecules in a population, e.g., a given PEGylated IFN-α species represents about 0.5%, about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or about 99.5% of the total population of IFN-α polypeptide molecules in a population.

IFN-β

The term interferon-beta (“IFN-β”) includes IFN-β polypeptides that are naturally occurring; non-naturally-occurring IFN-β polypeptides; and analogs of naturally occurring or non-naturally occurring IFN-β that retain antiviral activity of a parent naturally-occurring or non-naturally occurring IFN-β.

Any of a variety of beta interferons can be delivered by the continuous delivery method of the present invention. Suitable beta interferons include, but are not limited to, naturally-occurring IFN-β; IFN-β1a, e.g., Avonex® (Biogen, Inc.), and Rebif® (Serono, SA); IFN-β1b (Betaseron®; Berlex); and the like.

The IFN-β formulation may comprise an N-blocked species, wherein the N-terminal amino acid is acylated with an acyl group, such as a formyl group, an acetyl group, a malonyl group, and the like. Also suitable for use is a consensus IFN-β.

IFN-β polypeptides can be produced by any known method. DNA sequences encoding IFN-β may be synthesized using standard methods. In many embodiments, IFN-β polypeptides are the products of expression of manufactured DNA sequences transformed or transfected into bacterial hosts, e.g., E. coli, or in eukaryotic host cells (e.g., yeast; mammalian cells, such as CHO cells; and the like). In these embodiments, the IFN-β is “recombinant IFN-α.” Where the host cell is a bacterial host cell, the IFN-β is modified to comprise an N-terminal methionine.

It is to be understood that IFN-β as described herein may comprise one or more modified amino acid residues, e.g., glycosylations, chemical modifications, and the like.

IFN-tau

The term interferon-tau includes IFN-tau polypeptides that are naturally occurring; non-naturally-occurring IFN-tau polypeptides; and analogs of naturally occurring or non-naturally occurring IFN-tau that retain antiviral activity of a parent naturally-occurring or non-naturally occurring IFN-tau.

Suitable tau interferons include, but are not limited to, naturally-occurring IFN-tau; Tauferon® (Pepgen Corp.); and the like.

The IFN-tau formulation may comprise an N-blocked species, wherein the N-terminal amino acid is acylated with an acyl group, such as a formyl group, an acetyl group, a malonyl group, and the like. Also suitable for use is a consensus IFN-tau.

IFN-tau polypeptides can be produced by any known method. DNA sequences encoding IFN-tau may be synthesized using standard methods. In many embodiments, IFN-tau polypeptides are the products of expression of manufactured DNA sequences transformed or transfected into bacterial hosts, e.g., E. coli, or in eulcaryotic host cells (e.g., yeast; mammalian cells, such as CHO cells; and the like). In these embodiments, the IFN-tau is “recombinant IFN-α.” Where the host cell is a bacterial host cell, the IFN-tau is modified to comprise an N-terminal methionine.

It is to be understood that IFN-tau as described herein may comprise one or more modified amino acid residues, e.g., glycosylations, chemical modifications, and the like.

IFN-ω

The term interferon-omega (“IFN-ω”) includes IFN-ω) polypeptides that are naturally occurring; non-naturally-occurring IFN-ω polypeptides; and analogs of naturally occurring or non-naturally occurring IFN-ω that retain antiviral activity of a parent naturally-occurring or non-naturally occurring IFN-ω.

Any known omega interferon can be delivered by the continuous delivery method of the present invention. Suitable IFN-ω include, but are not limited to, naturally-occurring IFN-ω; recombinant IFN-ω, e.g., Biomed 510 (BioMedicines); and the like.

IFN-ω may comprise an amino acid sequence as set forth in GenBank Accession No. NP_{—}002168; or AAA70091. The sequence of any known IFN-ω polypeptide may be altered in various ways known in the art to generate targeted changes in sequence. A variant polypeptide will usually be substantially similar to the sequences provided herein, i.e. will differ by at least one amino acid, and may differ by at least two but not more than about ten amino acids. The sequence changes may be substitutions, insertions or deletions. Conservative amino acid substitutions typically include substitutions within the following groups: (glycine, alanine); (valine, isoleucine, leucine); (aspartic acid, glutamic acid); (asparagine, glutamine); (serine, threonine); (Oysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

The IFN-ω formulation may comprise an N-blocked species, wherein the N-terminal amino acid is acylated with an acyl group, such as a formyl group, an acetyl group, a malonyl group, and the like. Also suitable for use is a consensus IFN-ω.

IFN-ω polypeptides can be produced by any known method. DNA sequences encoding IFN-ω may be synthesized using standard methods. In many embodiments, IFN-ω polypeptides are the products of expression of manufactured DNA sequences transformed or transfected into bacterial hosts, e.g., E. coli, or in eukaryotic host cells (e.g., yeast; mammalian cells, such as CHO cells; and the like). In these embodiments, the IFN-ω) is “recombinant IFN-ω.” Where the host cell is a bacterial host cell, the IFN-ω is modified to comprise an N-terminal methionine.

It is to be understood that IFN-ω as described herein may comprise one or more modified amino acid residues, e.g., glycosylations, chemical modifications, and the like.

Agonists of Type II Interferon Receptors

In any of the above-described methods or apparatus, the interferon receptor agonist is in some embodiments an agonist of a Type II interferon receptor (e.g., “a Type II interferon agonist”). Type II interferon receptor agonists include an IFN-γ; antibody agonists specific for a Type II interferon receptor; and any other agonist of Type II interferon receptor, including non-polypeptide agonists.

IFN-γ

The nucleic acid sequences encoding IFN-γ polypeptides may be accessed from public databases, e.g. Genbank, journal publications, etc. While various mammalian IFN-γ polypeptides are of interest, for the treatment of human disease, generally the human protein will be used. Human IFN-γ coding sequence may be found in Genbank, accession numbers X13274; V00543; and NM_{—}000619. The corresponding genomic sequence may be found in Genbank, accession numbers J00219; M37265; and V00536. See, for example. Gray et al. (1982) Nature 295:501 (Genbank X13274); and Rinderknecht et al. (1984) J.B.C. 259:6790.

IFN-γ (Actimmune®; human interferon) is a single-chain polypeptide of 140 amino acids. It is made recombinantly in E. coli and is unglycosylated. Rinderknecht et al. (1984) J. Biol. Chem. 259:6790-6797.

The IFN-γ to be used in the compositions of the present invention may be any of natural IFN-γs, recombinant IFN-γs and the derivatives thereof so far as they have a IFN-γ activity, particularly human IFN-γ activity. Human IFN-γ exhibits the antiviral and anti-proliferative properties characteristic of the interferons, as well as a number of other immunomodulatory activities, as is known in the art. Although IFN-γ is based on the sequences as provided above, the production of the protein and proteolytic processing can result in processing variants thereof. The unprocessed sequence provided by Gray et al., supra. consists of 166 amino acids (aa). Although the recombinant IFN-γ produced in E. coli was originally believed to be 146 amino acids, (commencing at amino acid 20) it was subsequently found that native human IFN-γ is cleaved after residue 23, to produce a 143 aa protein, or 144 aa if the terminal methionine is present, as required for expression in bacteria. During purification, the mature protein can additionally be cleaved at the C terminus after reside 162 (referring to the Gray et al. sequence), resulting in a protein of 139 amino acids, or 140 amino acids if the initial methionine is present, e.g. if required for bacterial expression. The N-terminal methionine is an artifact encoded by the mRNA translational “start” signal AUG which, in the particular case of E. coli expression is not processed away. In other microbial systems or eukaryotic expression systems, methionine may be removed.

For use in the subject methods, any of the native IFN-γ peptides, modifications and variants thereof, or a combination of one or more peptides may be used. IFN-γ peptides of interest include fragments, and can be variously truncated at the carboxy terminal end relative to the full sequence. Such fragments continue to exhibit the characteristic properties of human gamma interferon, so long as-amino acids 24 to about 149 (numbering from the residues of the unprocessed polypeptide) are present. Extraneous sequences can be substituted for the amino acid sequence following amino acid 155 without loss of activity. See, for example, U.S. Pat. No. 5,690,925, herein incorporated by reference. Native IFN-γ moieties include molecules variously extending from amino acid residues 24-150; 24-151, 24-152; 24-153, 24-155; and 24-157. Any of these variants, and other variants known in the art and having IFN-γ activity, may be used in the present methods.

The sequence of the IFN-γ polypeptide may be altered in various ways known in the art to generate targeted changes in sequence. A variant polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

Included in the subject invention are polypeptides that have been modified using ordinary chemical techniques so as to improve their resistance to proteolytic degradation, to optrmize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g. D-amino acids or non-naturally occurring synthetic amino acids. The protein may be pegylated to enhance stability.

The polypeptides may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification-technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Agonists of Type III Interferon Receptors

In any of the above-described methods or apparatus, the interferon receptor agonist is in some embodiments an agonist of a Type III interferon receptor (e.g., “a Type II interferon agonist”). Type m interferon receptor agonists include an IL-28b polypeptide; and IL-28a polypeptide; and IL-29 polypeptide; antibody agonists specific for a Type III interferon receptor; and any other agonist of Type III interferon receptor, including non-polypeptide agonists.

IL-28A, IL-28B, and IL-29 (referred to herein collectively as “Type m interferons” or “Type III IFNs”) are described in Sheppard et al. (2003) Nature 4:63-68. Each polypeptide binds a heterodimeric receptor consisting of IL-10 receptor β chain and an IL-28 receptor α. Sheppard et al. (2003), supra. The amino acid sequences of IL-28A, IL-28B, and IL-29 are found under GenBank Accession Nos. NP_{—}742150, NP-742151, and NP_{—}742152, respectively.

The amino acid sequence of a Type III IFN polypeptide may be altered in various ways known in the art to generate targeted changes in sequence. A variant polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

Included in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g. D-amino acids or non-naturally occurring synthetic amino acids. The protein may be pegylated to enhance stability. The polypeptides may be fused to albumin.

The polypeptides may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

Dosages, Formulations, and Routes of Administration

An interferon receptor agonist and pirfenidone or pirfenidone analogs are administered to individuals in a formulation (e.g., in separate formulations) with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

In the subject methods, the active agent(s) may be administered to the host using any convenient means capable of resulting in the desired therapeutic effect. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the agents can be achieved in various ways, including oral, buccal, rectal, parenteral, intraperitoneal, intradermal, subcutaneous, intramuscular, transdermal, intratracheal, etc., administration. In some embodiments, two different routes of administration are used. For example, in some embodiments, an interferon receptor agonist, e.g., an IFN-α, is administered subcutaneously, and pirfenidone or pirfenidone analog is administered orally.

The route of administration of the interferon receptor agonist will depend in part on the interferon receptor agonist being administered. For example, an IFN-α is generally administered subcutaneously, by continuous delivery, or by a combination of subcutaneous (e.g., bolus injection) and continuous delivery. As another example, BETASERON® IFN-β1b is generally administered by subcutaneous injection. As another example, IFN-tau is generally administered orally. As another example, AVONEX® IFN-β1a is generally administered by intramuscular injection.

Subcutaneous administration of an interferon receptor agonist is accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of an interferon receptor agonist to a patient through the port is referred to herein as “a subcutaneous injection port delivery system.” In some embodiments, subcutaneous administration is achieved by a combination of devices, e.g., bolus delivery by needle and syringe, followed by delivery using a continuous delivery system.

In some embodiments, the interferon receptor agonist is delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, the present methods of drug delivery can be accomplished using any of a variety of refillable, pump systems. Pumps provide consistent, controlled release over time. Typically, the agent (e.g., interferon receptor agonist) is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are generally preferred because of convenience in implantation and removal of the drug delivery device.

Drug release devices suitable for use in the invention may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present invention. Examples of such devices include those described in, for example, U.S. Pat. Nos. 4,692,147; 4,360,019; 4,487,603; 4,360,019; 4,725,852, and the like. In general, the present methods of drug delivery can be accomplished using any of a variety of refillable, non-exchangeable pump systems. Pumps and other convective systems are generally preferred due to their generally more consistent, controlled release over time. Osmotic pumps are particularly preferred due to their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted infra, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, an interferon receptor agonist is delivered using an implantable drug delivery system, e.g., a system that is programmable to provide for administration of the interferon receptor agonist. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present invention is the Synchromed infusion pump (edtronic).

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the administered agent is an interferon receptor agonist polypeptide, a polynucleotide encoding the interferon receptor agonist may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the therapeutic DNA, then bombarded into skin cells. Of particular interest in these embodiments is use of a liver-specific promoter to drive transcription of an operably linked interferon receptor agonist coding sequence preferentially in liver cells.

In some embodiments, pirfenidone or a pirfenidone analog is administered during the entire course of interferon receptor agonist treatment. In other embodiments, pirfenidone or a pirfenidone analog is administered for a period of time that is overlapping with that of the interferon receptor agonist treatment, e.g., the pirfenidone or pirfenidone analog treatment can begin before the interferon receptor agonist treatment begins and end before the interferon receptor agonist treatment ends; the pirfenidone or pirfenidone analog treatment can begin after the interferon receptor agonist treatment begins and end after the interferon receptor agonist treatment ends; the pirfenidone or pirfenidone analog treatment can begin after the interferon receptor agonist treatment begins and end before the interferon receptor agonist treatment ends; or the pirfenidone or pirfenidone analog treatment can begin before the interferon receptor agonist treatment begins and end after the interferon receptor agonist treatment ends.

In connection with each of the methods described herein, the invention provides embodiments in which the interferon receptor agonist is administered to the patient by a controlled drug delivery device. In some embodiments, the interferon receptor agonist is delivered to the patient substantially continuously or continuously by the controlled drug delivery device. Optionally, an implantable infusion pump is used to deliver the interferon receptor agonist to the patient substantially continuously or continuously by subcutaneous infusion.

In other embodiments, the interferon receptor agonist is administered to the patient so as to achieve and maintain a desired average daily serum concentration of the interferon receptor agonist at a substantially steady state for the duration of the interferon receptor agonist therapy. Optionally, an implantable infusion pump is used to deliver the interferon receptor agonist to the patient by subcutaneous infusion so as to achieve and maintain a desired average daily serum concentration of the interferon receptor agonist at a substantially steady state for the duration of the interferon receptor agonist therapy.

Where the interferon receptor agonist is an IFN-α, in general, effective dosages of IFN-α can range from 0.3 μg to 100 μg. Effective dosages of Infergen®consensus IFN-α contain an amount of about 3 μg, about 9 μg, about 15 μg, about 18 μg, or about 27 μg of drug per dose. Effective dosages of IFN-α2a and IFN-α2b contain an amount of about 3 million Units (MU) to about 10 MU of drug per dose. Effective dosages of PEGASYS®PEGylated IFN-α2a contain an amount of about 90 μg to about 180 μg, or about 135 μg, of drug per dose. Effective dosages of PEG-INTRON®PEGylated IFN-α2b contain an amount of about 0.5 μg to about 1.5 μg of drug per kg of body weight per dose. Effective dosages of PEGylated consensus interferon (PEG-CIFN) contain an amount of about 18 μg to about 90 μg, or about 27 μg to about 60 μg, or about 45 μg, of CIFN amino acid weight per dose of PEG-CIFN.

Where the interferon receptor agonist is an IFN-β, in general, effective dosages of IFN-β can range from 3 μg to about 300 μg. Exemplary effective dosages of an IFN-β are 30 μg, 44 μg, and 300 μg.

Where the interferon receptor agonist is an IFN-γ, suitable dosages of IFN-γ can range from about 25 μg/dose to about 300 μg/dose, or from about 100 μg/dose to about 1,000 μg/dose.

In many embodiments, the interferon receptor agonist and/or pirfenidone or pirfenidone analog is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The interferon receptor agonist can be administered tid, bid, qd, qod, biw, tiw, qw, qow, three times per month, once monthly, substantially continuously, or continuously.

Those of skill will readily appreciate that dose levels can vary as a function of the specific interferon receptor agonist, the severity of the symptoms and the susceptibility of the subject to side effects. Preferred dosages for a given interferon receptor agonist are readily determinable by those of skill in the art by a variety of means. A preferred means is to measure the physiological potency of a given interferon receptor agonist.

In many embodiments, multiple doses of interferon receptor agonist are administered. For example, an interferon receptor agonist is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), substantially continuously, or continuously, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In general, effective dosages of pirfenidone or specific pirfenidone analogs can range from about 0.5 mg/kg/day to about 200 mg/kg/day, or at a fixed dosage of about 400 mg to about 3600 mg per day, or about 50 mg to about 5,000 mg per day, or about 100 mg to about 1,000 mg per day, administered orally, optionally in two or more divided doses per day. Other doses and formulations of pirfenidone and pirfenidone analogs suitable for use in the treatment of an alphavirus infection are described in U.S. Pat. Nos. 3,974,281; 3,839,346; 4,042,699; 4,052,509; 5,310,562; 5,518,729; 5,716,632; and 6,090,822.

Those of skill in the art will readily appreciate that dose levels of pirfenidone or pirfenidone analog can vary as a function of the specific compound, the severity of the symptoms and the susceptibility of the subject to side effects. Preferred dosages for a given compound are readily determinable by those of skill in the art by a variety of means.

Pirfenidone (or a pirfenidone analog) can be administered daily, twice a day, or three times a day, or in divided daily doses ranging from 2 to 5 times daily over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

An interferon receptor agonist and pirfenidone (or pirfenidone analog) are generally administered in separate formulations. An interferon receptor agonist and pirfenidone (or pirfenidone analog) may be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

1. Treatment of Alphaviral Infections

The present invention provides methods of treating alphaviral infection by administering a therapeutically effective amount of an interferon receptor agonist and pirfenidone or a pirfenidone analog to an individual in need thereof. Individuals who are to be treated according to the methods of the invention include individuals who have been clinically diagnosed with an alphaviral infection, as well as individuals who exhibit one or more of the signs and the symptoms of clinical infection but have not yet been diagnosed with an alphaviral infection.

Low Dose Interferon Receptor Agonist in Synergistic Combination with Pirfenidone

In some embodiments, the invention provides methods using a synergistically effective amount of an interferon receptor agonist and pirfenidone or a pirfenidone analog in the treatment of alpliaviral infection in a patient. In these embodiments, a low dose of an interferon receptor agonist is administered in combination therapy with pirfenidone or a pirfenidone analog. In particular embodiments, the invention provides a method using a synergistically effective amount of an IFN-α and pirfenidone or a pirfenidone analog in the treatment of alphaviral infection in a patient. In one embodiment, the invention provides a method using a synergistically effective amount of a consensus IFN-α and pirfenidone or a pirfenidone analog in the treatment of alphaviral infection in a patient.

In another embodiment, the invention provides a method using a synergistically effective amount of INFERGEN®consensus IFN-α and pirfenidone or a specific pirfenidone analog in the treatment of alphaviral infection in a patient comprising administering to the patient a dosage of INFERGEN® containing an amount of about 1 μg to about 30 μg, of drug per dose of INFERGEN®, subcutaneously qd, qod, tiw, or biw, or per day substantially continuously or continuously, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In another embodiment, the invention provides a method using a synergistically effective amount of INFERGEN®consensus IFN-α and pirfenidone or a specific pirfenidone analog in the treatment of alphaviral infection in a patient comprising administering to the patient a dosage of INFERGEN® containing an amount of about 1 μg to about 9 μg, of drug per dose of INFERGEN®, subcutaneously qd, qod, tiw, or biw, or per day substantially continuously or continuously, in combination with a dosage of pirfenidone or a specific pirfenidone analog containing an amount of about 100 mg to about 1,000 mg of drug per dose of pirfenidone or a specific pirfenidone analog orally qd, optionally in two or more divided doses per day, for the desired treatment duration.

In another embodiment, the invention provides a method using a synergistically effective amount of INFERGEN®consensus IFN-α and pirfenidone or a specific pirfenidone analog in the treatment of alphaviral infection in a patient comprising administering to the patient a dosage of INFERGEN® containing an amount of about 9 μg, of drug per dose of IFERGEN®, subcutaneously qd, qod, tiw, or biw, or per day substantially continuously or continuously, in combination with a dosage of pirfenidone or a specific pirfenidone analog containing an amount of about 500 mg of drug per dose of pirfenidone or a specific pirfenidone analog orally qd, optionally in two or more divided doses per day, for the desired treatment duration.

In another embodiment, the invention provides a method using a synergistically effective amount of a consensus IFN-α and pirfenidone or a specific pirfenidone analog in the treatment of alphaviral infection in a patient comprising administering to the patient a dosage of PEGylated consensus IFN-α (PEG-CFN) containing an amount of about 10 μg to about 150 μg of CIFN amino acid weight per dose of PEG-CIFN, subcutaneously qw, qow, three times per month, or monthly, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In another embodiment, the invention provides a method using a synergistically effective amount of a consensus IFN-α and pirfenidone or a specific pirfenidone analog in the treatment of alphaviral infection in a patient comprising administering to the patient a dosage of PEGylated consensus IFN-α (PEG-CIFN) containing an amount of about 45 μg to about 60 μg of CIFN amino acid weight per dose of PEG-CIFN, subcutaneously qw, qow, three times per month, or monthly, in combination with a dosage of pirfenidone or a specific pirfenidone analog containing an amount of about 10 mg to about 1,000 mg of drug per dose of pirfenidone or a specific pirfenidone analog orally qd, optionally in two or more divided doses per day, for the desired treatment duration.

In another embodiment, the invention provides a method using a synergistically effective amount of a consensus IFN-α and pirfenidone or a specific pirfenidone analog in the treatment of alphaviral infection in a patient comprising administering to the patient a dosage of PEGylated consensus IFN-α (PEG-CIFN) containing an amount of about 45 μg to about 60 μg of CIFN amino acid weight per dose of PEG-CIFN, subcutaneously qw, qow, three times per month, or monthly, in combination with a dosage of pirfenidone or a specific pirfenidone analog containing an amount of about 500 mg of drug per dose of pirfenidone or a specific pirfenidone analog orally qd, optionally in two or more divided doses per day, for the desired treatment duration.

In another embodiment, the invention provides a method using a synergistically effective amount of IFN-α 2a or 2b or 2c and pirfenidone or a specific pirfenidone analog in the treatment of alphaviral infection in a patient comprising administering to the patient a dosage of IFN-α 2a or 2b or 2c containing an amount of about 1 MU to about 20 MU, of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In another embodiment, the invention provides a method using a synergistically effective amount of IFN-α 2a or 2b or 2c and pirfenidone or a specific pirfenidone analog in the treatment of alphaviral infection in a patient comprising administering to the patient a dosage of IFN-α 2a or 2b or 2c containing an amount of about 3 MU to about 10 MU of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount about 100 of mg to about 1,000 mg of drug per dose of pirfenidone or a specific pirfenidone analog orally qd, optionally in two or more divided doses per day, for the desired treatment duration.

In another embodiment, the invention provides a method using a synergistically effective amount of IFN-α 2a or 2b or 2c and pirfenidone or a specific pirfenidone analog in the treatment of alphaviral infection in a patient comprising administering to the patient a dosage of IFN-α 2a or 2b or 2c containing an amount of about 3 MU of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount about 500 mg of drug per dose of pirfenidone or a specific pirfenidone analog orally qd, optionally in two or more divided doses per day, for the desired treatment duration.

In another embodiment, the invention provides a method using a synergistically effective amount of PEGASYS®PEGylated IFN-α2a and pirfenidone or a specific pirfenidone analog in the treatment of alphaviral infection in a patient comprising administering to the patient a dosage of PEGASYS® containing an amount of about 90 μg to about 360 μg of drug per dose of PEGASYS®, subcutaneously qw, qow, three times per month, or monthly, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In another embodiment, the invention provides a method using a synergistically effective amount of PEGASYS® PEGylated IFN-α2a and pirfenidone or a specific pirfenidone analog in the treatment of alphaviral infection in a patient comprising administering to the patient a dosage of PEGASYS® containing an amount of about 180 μg of drug per dose of PEGASYS®, subcutaneously qw, qow, three times per month, or monthly, in combination with a dosage of pirfenidone or a specific pirfenidone analog containing an amount of about 500 mg of drug per dose of pirfenidone or a specific pirfenidone analog orally qd, optionally in two or more divided doses per day, for the desired treatment duration.

In another embodiment, the invention provides a method using a synergistically effective amount of PEG-INTRON® PEGylated IFN-α2b and pirfenidone or a specific pirfenidone analog in the treatment of alphaviral infection in a patient comprising administering to the patient a dosage of PEG-INTRON® containing an amount of about 0.75 jig to about 3.0 μg of drug per kilogram of body weight per dose of PEG-INTRON®, subcutaneously qw, qow, three times per month, or monthly, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per month, for the desired treatment duration.

In another embodiment, the invention provides a method using a synergistically effective amount of PEG-INTRON® PEGylated IFN-α2b and pirfenidone or a specific pirfenidone analog in the treatment of alphaviral infection in a patient comprising administering to the patient a dosage of PEG-INTRON® containing an amount of about 1.5 μg of drug per kilogram of body weight per dose of PEG-INTRON®, subcutaneously qw, qow, three times per month, or monthly, in combination with a dosage of pirfenidone or a specific pirfenidone analog containing an amount of about 500 mg of drug per dose of pirfenidone or a specific pirfenidone analog orally qd, optionally in two or more divided doses per month, for the desired treatment duration.

High Dose IFN-α in Combination with Pirfenidone

In addition to synergistic combinations of an interferon receptor agonist and pirfenidone or a pirfenidone analog, combination therapy involving administering a high dose of an interferon receptor agonist and an effective amount of pirfenidone or a pirfenidone analog is provided. Pirfenidone can reduce undesirable side effects of the interferon receptor agonist, thus permitting the use of higher doses.

In some of these embodiments, the interferon receptor agonist is administered at or near, or even exceeding the maximum tolerated dose (MTD). In this context, the term “MTD” refers to the maximum amount of the interferon receptor agonist tolerated by the patient in interferon receptor agonist monotherapy. For example, the term “MID,” in the context of IFN-α, refers to the maximum amount of IFN-α tolerated by the patient in IFN-α monotherapy.

In another embodiment, the invention provides a method using an effective amount of INFERGENαconsensus IFN-α and pirfenidone or a specific pirfenidone analog in the treatment of alphaviral infection in a patient comprising administering to the patient a dosage of INFERGENα containing an amount of about 5 μg to about 150 μg, of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 10,000 mg of drug per dose of pirfenidone or a specific pirfenidone analog orally qd, optionally in two or more divided doses per day, for the desired treatment duration.

In another embodiment, the invention provides a method using an effective amount of INFERGEN®consensus IFN-α and pirfenidone or a specific pirfenidone analog in the treatment of alphaviral infection in a patient comprising administering to the patient a dosage of INFERGEN® containing an amount of about 5 μg to about 45 μg, of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 3,000 mg of drug per dose of pirfenidone or a specific pirfenidone analog orally qd, optionally in two or more divided doses per day, for the desired treatment duration.

In another embodiment, the invention provides a method using an effective amount of INFERGEN®consensus IFN-α and pirfenidone or a specific pirfenidone analog in the treatment of alphaviral infection in a patient comprising administering to the patient a dosage of INFERGEN® containing an amount of about 45 μg, of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose of pirfenidone or a specific pirfenidone analog orally qd, optionally in two or more divided doses per day, for the desired treatment duration.

In another embodiment, the invention provides a method using an effective amount of a consensus IFN-α and pirfenidone or a specific pirfenidone analog in the treatment of alphaviral infection in a patient comprising administering to the patient a dosage of PEGylated consensus IFN-α (PEG-CIFN) containing an amount of about 50 μg to about 750 μg of CIFN amino acid weight per dose of PEG-CIFN, subcutaneously qw, qow, three times per month, or monthly, in combination with a dosage of pirfenidone or a specific pirfenidone analog containing an amount of about 1,000 mg to about 10,000 mg of drug per dose of pirfenidone or a specific pirfenidone analog orally qd, optionally in two or more divided doses per day, for the desired treatment duration.

In another embodiment, the invention provides a method using an effective amount of a consensus IFN-α and pirfenidone or a specific pirfenidone analog in the treatment of alphaviral infection in a patient comprising administering to the patient a dosage of PEGylated consensus IFN-α (PEG-CIFN) containing an amount of about 225 μg to about 300 μg of CIFN amino acid weight per dose of PEG-CIFN, subcutaneously qw, qow, three times per month, or monthly, in combination with a dosage of pirfenidone or a specific pirfenidone analog containing an amount of about 1,000 mg to about 3,000 mg of drug per dose of pirfenidone or a specific pirfenidone analog orally qd, optionally in two or more divided doses per day, for the desired treatment duration.

In another embodiment, the invention provides a method using an effective amount of a consensus IFN-α and pirfenidone or a specific pirfenidone analog in the treatment of alphaviral infection in a patient comprising administering to the patient a dosage of PEGylated consensus IFN-α (PEG-CIFN) containing an amount of about 225 μg to about 300 μg of CIFN amino acid weight per dose of PEG-CIFN, subcutaneously qw, qow, three times per month, or monthly, in combination with a dosage of pirfenidone or a specific pirfenidone analog containing an amount of about 1,000 mg to about 2,000 mg of drug per dose of pirfenidone or a specific pirfenidone analog orally qd, optionally in two or more divided doses per day, for the desired treatment duration.

In another embodiment, the invention provides a method using an effective amount of IFN-α 2a or 2b or 2c and pirfenidone or a specific pirfenidone analog in the treatment of alphaviral infection in a patient comprising administering to the patient a dosage of IFN-α 2a or 2b or 2c containing an amount of about 5 MU to about 100 MU, of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount of about 1,000 mg to about 10,000 mg of drug per dose of pirfenidone or a specific pirfenidone analog orally qd, optionally in two or more divided doses per day, for the desired treatment duration.

In another embodiment, the invention provides a method using an effective amount of IFN-α 2a or 2b or 2c and pirfenidone or a specific pirfenidone analog in the treatment of alphaviral infection in a patient comprising administering to the patient a dosage of IFN-α 2a or 2b or 2c containing an amount of about 15 MU to about 50 MU of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount about 1,000 of mg to about 3,000 mg of drug per dose of pirfenidone or a specific pirfenidone analog orally qd, optionally in two or more divided doses per day, for the desired treatment duration.

In another embodiment, the invention provides a method using an effective amount of IFN-α 2a or 2b or 2c and pirfenidone or a specific pirfenidone analog in the treatment of alphaviral infection in a patient comprising administering to the patient a dosage of IFN-α 2a or 2b or 2c containing an amount of about 15 MU of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount about 1,000 mg to about 2,000 mg of drug per dose of pirfenidone or a specific pirfenidone analog orally qd, optionally in two or more divided doses per day, for the desired treatment duration.

In another embodiment, the invention provides a method using an effective amount of PEGASYS® PEGylated IFN-α2a and pirfenidone or a specific pirfenidone analog in the treatment of alphaviral infection in a patient comprising administering to the patient a dosage of PEGASYS® containing an amount of about 450 μg to about 1800 μg of drug per dose of PEGASYS®, subcutaneously qw, qow, three times per month, or monthly, in combination with a dosage of pirfenidone or a specific pirfenidone analog containing an amount of about 1,000 mg to about 10,000 mg of drug per dose of pirfenidone or a specific pirfenidone analog orally qd, optionally in two or more divided doses per day, for the desired treatment duration.

In another embodiment, the invention provides a method using an effective amount of PEGASYS® PEGylated IFN-α2a and pirfenidone or a specific pirfenidone analog in the treatment of alphaviral infection in a patient comprising administering to the patient a dosage of PEGASYS® containing an amount of about 900 μg of drug per dose of PEGASYS®, subcutaneously qw, qow, three times per month, or monthly, in combination with a dosage of pirfenidone or a specific pirfenidone analog containing an amount of about 1,000 mg to about 2,000 mg of drug per dose of pirfenidone or a specific pirfenidone analog orally qd, optionally in two or more divided doses per day, for the desired treatment duration.

In another embodiment, the invention provides a method using an effective amount of PEG-INTRON® PEGylated IFN-α2b and pirfenidone or a specific pirfenidone analog in the treatment of alphaviral infection in a patient comprising administering to the patient a dosage of PEG-INTRON® containing an amount of about 0.375 μg to about 15.0 μg of drug per kilogram of body weight per dose of PEG-INTRON®, subcutaneously qw, qow, three times per month, or monthly, in combination with a dosage of pirfenidone or a specific pirfenidone analog containing an amount of about 1,000 mg to about 10,000 mg of drug per dose of pirfenidone or a specific pirfenidone analog orally qd, optionally in two or more divided doses per month, for the desired treatment duration.

In another embodiment, the invention provides a method using an effective amount of PEG-INTRON® PEGylated IFN-α2b and pirfenidone or a specific pirfenidone analog in the treatment of alphaviral infection in a patient comprising administering to the patient a dosage of PEG-INTRON® containing an amount of about 7.5 μg of drug per kilogram of body weight per dose of PEG-INTRON®, subcutaneously qw, qow, three times per month, or monthly, in combination with a dosage of pirfenidone or a specific pirfenidone analog containing an amount of about 1,000 mg to about 2,000 mg of drug per dose of pirfenidone or a specific pirfenidone analog orally qd, optionally in two or more divided doses per month, for the desired treatment duration.

Combination Therapy with Ribavirin

The invention also provides methods for the treatment of alphaviral infection in which ribavirin therapy is added to any of the interferon receptor agonist and pirfenidone or a pirfenidone analog combination therapies described above. In some embodiments, the interferon receptor agonist and pirfenidone or a pirfenidone analog combination therapy is modified to include a ribavirin regimen of 800 mg to 1200 mg ribavirin orally qd for the specified duration of therapy. In other embodiments, the interferon receptor agonist and pirfenidone or a pirfenidone analog combination therapy is modified to include a ribavirin regimen of 1000 mg ribavirin orally qd for the specified duration of therapy. In additional embodiments, the interferon receptor agonist and pirfenidone or a pirfenidone analog combination therapy is modified to include a ribavirin regimen of about 10 mg of ribavirin/kg body weight orally qd for the specified duration of therapy. The daily ribavirin dosage can be administered in one dose per day or in divided doses, including one, two, three or four doses, per day.

2. Treatment of HCV Infections

The present invention provides methods of treating hepatitis C virus infection by administering a therapeutically effective amount of an interferon receptor agonist and pirfenidone or a pirfenidone analog to an individual in need thereof. Individuals who are to be treated according to the methods of the invention include individuals who have been clinically diagnosed with an HCV infection, as well as individuals who exhibit one or more of the signs and the symptoms of clinical infection but have not yet been diagnosed with an HCV infection.

Individuals who are clinically diagnosed as infected with HCV include naive individuals (e.g., individuals not previously treated for HCV) and individuals who have failed prior treatment for HCV (“treatment failure” patients). Treatment failure patients include non-responders (e.g., individuals in whom the HCV titer was not significantly or sufficiently reduced by a previous treatment for HCV); and relapsers (e.g., individuals who were previously treated for HCV, whose HCV titer decreased, and subsequently increased).

In particular embodiments of interest, individuals have an HCV titer of at least about 10⁵, at least about 5×10⁵, or at least about 10⁶, or at least about 2×10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1 and particular HCV subtypes and quasispecies.

Also of interest are HCV-positive individuals (as described above) who exhibit severe fibrosis or early cirrhosis (non-decompensated, Child's-Pugh class A or less), or more advanced cirrhosis (decompensated, Child's-Pugh class B or C) due to chronic HCV infection and who are viremic despite prior anti-viral treatment with IFN-α-based therapies or who cannot tolerate IFN-α-based therapies, or who have a contraindication to such therapies. In particular embodiments of interest, HCV-positive individuals with stage 3 or 4 liver fibrosis according to the METAVIR scoring system are suitable for treatment with the methods of the present invention. In other embodiments; individuals suitable for treatment with the methods of the instant invention are patients with decompensated cirrhosis with clinical manifestations, including patients with far-advanced liver cirrhosis, including those awaiting liver transplantation. In still other embodiments, individuals suitable for treatment with the methods of the instant invention include patients with milder degrees of fibrosis including those with early fibrosis (stages 1 and 2 in the METAVIR, Ludwig, and Scheuer scoring systems; or stages 1, 2, or 3 in the Ishak scoring system.).

In carrying out the methods of combination therapy for hepatitis C viral infection in an individual as described above, an interferon receptor agonist and pirfenidone or a pirfenidone analog are administered to the individual. In general, the interferon receptor agonist and pirfenidone or a pirfenidone analog are administered in separate formulations. When administered in separate formulations, the interferon receptor agonist and pirfenidone or a pirfenidone analog can be administered substantially simultaneously, or can be administered within about 24 hours of one another. In many embodiments, the interferon receptor agonist and pirfenidone or a pirfenidone analog are administered subcutaneously in multiple doses.

Effective weight-based dosages or pirfenidone or specific pirfenidone analog generally range from about 5 mg/kg of body weight to about 175 mg/kg of body weight orally qd for the duration of the desired interferon receptor agonist therapy. Effective fixed dosages of pirfenidone or specific pirfenidone analog range from about 400 mg to about 3600 mg, or about 800 mg to about 2400 mg, or about 1000 mg to about 1800 mg, or about 1200 mg to about 1600 mg, orally qd for the duration of the interferon receptor agonist therapy.

Effective dosages of IFN-α generally range from about 3 μg/dose to about 135 μg/dose. Effective dosages of Infergen®consensus IFN-α contain an amount of about 3 μg, about 9 μg, about 15 μg, about 18 μg, or about 27 μg, of drug per dose. Effective dosages of IFN-α2a and IFN-α2b contain an amount of about 3 million Units (MU) to about 10 MU of drug per dose. Effective dosages of PEGASYS®PEGylated IFN-α2a contain an amount of about 90 μg to about 180 μg, or about 135 μg, of drug per dose. Effective dosages of PEG-INTRON® PEGylated IFN-α2b contain an amount of about 0.5 μg to about 1.5 μg of drug per kg body weight per dose. Effective dosages of PEGylated consensus interferon (PEG-CIFN) contain an amount of about 18 μg to about 90 μg, or about 27 μg to about 60 μg, or about 45 μg, of CIFN amino acid weight per dose of PEG-CIFN.

Where the interferon receptor agonist is an IFN-β, in general, effective dosages of IFN-β can range from 3 μg to about 300 μg. Exemplary effective dosages of an IFN-β are 30 μg, 44 μg, and 300 μg.

Where the interferon receptor agonist is an IFN-γ, suitable dosages of IFN-γ range from about 25 μg/dose to about 300 μg/dose.

In many embodiments, the interferon receptor agonist and pirfenidone or a pirfenidone analog are administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. Dosage regimens can include tid, bid, qd, qod, biw, tiw, qw, qow, three times per month, or monthly administrations.

In certain embodiments, the specific regimen of drug therapy used in treatment of the HCV patient is selected according to certain disease parameters exhibited by the patient, such as the initial viral load, genotype of the HCV infection in the patient, liver histology and/or stage of liver fibrosis in the patient In one embodiment, the invention provides a method for treatment of HCV infection comprising the steps of (1) identifying a patient having advanced or severe stage liver fibrosis as measured by a Knodell score of 3 or 4 and then (2) administering to the patient a therapeutically effective amount of an interferon receptor agonist and pirfenidone or a pirfenidone analog for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

In another embodiment, the invention provides a method for treatment of HCV infection comprising the steps of (1) identifying a patient having advanced or severe stage liver fibrosis as measured by a Knodell score of 3 or 4 and then (2) administering to the patient a therapeutically effective amount of IFN-α and pirfenidone or a pirfenidone analog for a time period of about 40 weeks to about 50 weeks, or about 48 weeks.

In another embodiment, the invention provides a method for treatment of HCV infection comprising the steps of (1) identifying a patient having an HCV genotype 1 infection and an initial viral load of greater than 2 million viral genome copies per ml of patient serum and then (2) administering to the patient a therapeutically effective amount of an interferon receptor agonist and pirfenidone or a pirfenidone analog for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

In another embodiment, the invention provides a method for treatment of HCV infection comprising the steps of (1) identifying a patient having an HCV genotype 1 infection and an initial viral load of greater than 2 million viral genome copies per ml of patient serum and then (2) administering to the patient a therapeutically effective amount of an interferon receptor agonist and pirfenidone or a pirfenidone analog for a time period of about 40 weeks to about 50 weeks, or about 48 weeks.

In another embodiment, the invention provides a method for treatment of HCV infection comprising the steps of (1) identifying a patient having an HCV genotype 1 infection and an initial viral load of greater than 2 million viral genome copies per ml of patient serum and no or early stage liver fibrosis as measured by a Knodell score of 0, 1, or 2 and then (2) administering to the patient a therapeutically effective amount of IFN-α and pirfenidone or a pirfenidone analog for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

In another embodiment, the invention provides a method for treatment of HCV infection comprising the steps of (1) identifying a patient having an HCV genotype 1 infection and an initial viral load of greater than 2 million viral genome copies per ml of patient serum and no or early stage liver fibrosis as measured by a Knodell score of 0, 1, or 2 and then (2) administering to the patient a therapeutically effective amount of an interferon receptor agonist and pirfenidone or a pirfenidone analog for a time period of about 40 weeks to about 50 weeks, or about 48 weeks.

In another embodiment, the invention provides a method for treatment of HCV infection comprising the steps of (1) identifying a patient having an HCV genotype 1 infection and an initial viral load of less than or equal to 2 million viral genome copies per ml of patient serum and then (2) administering to the patient a therapeutically effective amount of an interferon receptor agonist and pirfenidone or a pirfenidone analog for a time period of about 20 weeks to about 50 weeks, or about 24 weeks to about 48 weeks, or about 30 weeks to about 40 weeks, or up to about 20 weeks, or up to about 24 weeks, or up to about 30 weeks, or up to about 36 weeks, or up to about 48 weeks.

In another embodiment, the invention provides a method for treatment of HCV infection comprising the steps of (1) identifying a patient having an HCV genotype 1 infection and an initial viral load of less than or equal to 2 million viral genome copies per ml of patient serum and then (2) administering to the patient a therapeutically effective amount of an interferon receptor agonist and pirfenidone or a pirfenidone analog for a time period of about 20 weeks to about 24 weeks.

In another embodiment, the invention provides a method for treatment of HCV infection comprising the steps of (1) identifying a patient having an HCV genotype 1 infection and an initial viral load of less than or equal to 2 million viral genome copies per ml of patient serum and then (2) administering to the patient a therapeutically effective amount of an interferon receptor agonist and pirfenidone or a pirfenidone analog for a time period of about 24 weeks to about 48 weeks.

In another embodiment, the invention provides a method for treatment of HCV infection comprising the steps of (1) identifying a patient having an HCV genotype 2 or 3 infection and then (2) administering to the patient a therapeutically effective amount of an interferon receptor agonist and pirfenidone or a pirfenidone analog for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

In another embodiment, the invention provides a method for treatment of HCV infection comprising the steps of (1) identifying a patient having an HCV genotype 2 or 3 infection and then (2) administering to the patient a therapeutically effective amount of an interferon receptor agonist and pirfenidone or a pirfenidone analog for a time period of about 20 weeks to about 50 weeks, or about 24 weeks to about 48 weeks, or about 30 weeks to about 40 weeks, or up to about 20 weeks, or up to about 24 weeks, or up to about 30 weeks, or up to about 36 weeks, or up to about 48 weeks.

In another embodiment, the invention provides a method for treatment of HCV infection comprising the steps of (1) identifying a patient having an HCV genotype 2 or 3 infection and then (2) administering to the patient a therapeutically effective amount of an interferon receptor agonist and pirfenidone or a pirfenidone analog for a time period of about 20 weeks to about 24 weeks.

In another embodiment, the invention provides a method for treatment of HCV infection comprising the steps of (1) identify a patient having an HCV genotype 2 or 3 infection and then (2) administering to the patient a therapeutically effective amount of an interferon receptor agonist and pirfenidone or a pirfenidone analog for a time period of at least about 24 weeks.

In another embodiment, the invention provides a method for treatment of HCV infection comprising the steps of (1) identifying a patient having an HCV genotype 4 infection and then (2) administering to the patient a therapeutically effective amount of an interferon receptor agonist and pirfenidone or a pirfenidone analog for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

In another embodiment, the invention provides a method for treatment of HCV infection comprising the steps of (1) identifying a patient having an HCV infection characterized by any of HCV genotypes 5, 6, 7, 8 and 9 and then (2) administering to the patient a therapeutically effective amount of an interferon receptor agonist and pirfenidone or a pirfenidone analog for a time period of about 20 weeks to about 50 weeks.

In another embodiment, the invention provides a method for treatment of HCV infection comprising the steps of (1) identifying a patient having an HCV infection characterized by any of HCV genotypes 5, 6, 7, 8 and 9 and then (2) administering to the patient a therapeutically effective amount of an interferon receptor agonist and pirfenidone or a pirfenidone analog for a time period of at least about 24 weeks and up to about 48 weeks.

Low Dose Interferon Receptor Agonist in Synergistic Combination with Pirfenidone

In some embodiments, the invention provides methods using a synergistically effective amount of interferon receptor agonist and pirfenidone or a pirfenidone analog in the treatment of an HCV infection in a patient. In these embodiments, a low dose of interferon receptor agonist is administered in combination therapy with pirfenidone or a pirfenidone analog. In some of these embodiments, the invention provides a method using a synergistically effective amount of a IFN-α and pirfenidone or a pirfenidone analog in the treatment of an HCV infection in a patient. In these embodiments, a low dose of IFN-α is administered in combination therapy with pirfenidone or a pirfenidone analog. In one embodiment, the invention provides a method using a synergistically effective amount of a consensus IFN-α and pirfenidone or a pirfenidone analog in the treatment of an HCV infection in a patient.

In another embodiment, the invention provides a method using a synergistically effective amount of INFERGEN®consensus IFN-α and pirfenidone or a specific pirfenidone analog in the treatment of HCV infection in a patient comprising administering to the patient a dosage of INFERGEN® continuing an amount of about 1 μg to about 30 μg, of drug per dose of INFERGEN®, subcutaneously qd, qod, tiw, or biw, or per day substantially continuously or continuously, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In another embodiment, the invention provides a method using a synergistically effective amount of INFERGEN®consensus IFN-α and pirfenidone or a specific pirfenidone analog in the treatment of HCV infection in a patient comprising administering to the patient a dosage of INFERGEN® containing an amount of about 1 μg to about 9 μg, of drug per dose of INFERGEN®, subcutaneously qd, qod, tiw, or biw, or per day substantially continuously or continuously, in combination with a dosage of pirfenidone or a specific pirfenidone analog containing an amount of about 100 mg to about 1,000 mg of drug per dose of pirfenidone or a specific pirfenidone analog orally qd, optionally in two or more divided doses per day, for the desired treatment duration.

In another embodiment, the invention provides a method using a synergistically effective amount of INFERGEN®consensus IFN-α and pirfenidone or a specific pirfenidone analog in the treatment of HCV infection in a patient comprising administering to the patient a dosage of INFERGEN® containing an amount of about 9 μg, of drug per dose of INFERGEN®, subcutaneously qd, qod, tiw, or biw, or per day substantially continuously or continuously, in combination with a dosage of pirfenidone or a specific pirfenidone analog containing an amount of about 500 mg of drug per dose of pirfenidone or a specific pirfenidone analog orally qd, optionally in two or more divided doses per day, for the desired treatment duration.

In another embodiment, the invention provides a method using a synergistically effective amount of a consensus IFN-α and pirfenidone or a specific pirfenidone analog in the treatment of HCV infection in a patient comprising administering to the patient a dosage of PEGylated consensus IFN-α (PEG-CIFN) containing an amount of about 10 μg to about 150 μg of CIFN amino acid weight per dose of PEG-CIFN, subcutaneously qw, qow, three times per month, or monthly, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In another embodiment, the invention provides a method using a synergistically effective amount of a consensus IFN-α and pirfenidone or a specific pirfenidone analog in the treatment of HCV infection in a patient comprising administering to the patient a dosage of PEGylated consensus IFN-α (PEG-CIFN) containing an amount of about 45 μg to about 60 μg of CIFN amino acid weight per dose of PEG-CIFN, subcutaneously qw, qow, three times per month, or monthly, in combination with a dosage of pirfenidone or a specific pirfenidone analog containing an amount of about 10 mg to about 1,000 mg of drug per dose of pirfenidone or a specific pirfenidone analog orally qd, optionally in two or more divided doses per day, for the desired treatment duration.

In another embodiment, the invention provides a method using a synergistically effective amount of a consensus IFN-α and pirfenidone or a specific pirfenidone analog in the treatment of HCV infection in a patient comprising administering to the patient a dosage of PEGylated consensus IFN-α (PEG-CIFN) containing an amount of about 45 μg to about 60 μg of CIFN amino acid weight per dose of PEG-CIFN, subcutaneously qw, qow, three times per month, or monthly, in combination with a dosage of pirfenidone or a specific pirfenidone analog containing an amount of about 500 mg of drug per dose of pirfenidone or a specific pirfenidone analog orally qd, optionally in two or more divided doses per day, for the desired treatment duration.

In another embodiment, the invention provides a method using a synergistically effective amount of IFN-α 2a or 2b or 2c and pirfenidone or a specific pirfenidone analog in the treatment of HCV infection in a patient comprising administering to the patient a dosage of IFN-α 2a or 2b or 2c containing an amount of about 1 MU to about 20 MU, of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In another embodiment, the invention provides a method using a synergistically effective amount of IFN-α 2a or 2b or 2c and pirfenidone or a specific pirfenidone analog in the treatment of HCV infection in a patient comprising administering to the patient a dosage of IFN-α 2a or 2b or 2c containing an amount of about 3 MU to about 10 MU of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount about 100 of mg to about 1,000 mg of drug per dose of pirfenidone or a specific pirfenidone analog orally qd, optionally in two or more divided doses per day, for the desired treatment duration.

In another embodiment, the invention provides a method using a synergistically effective amount of IFN-α 2a or 2b or 2c and pirfenidone or a specific pirfenidone analog in the treatment of HCV infection in a patient comprising administering to the patient a dosage of IFN-α 2a or 2b or 2c containing an amount of about 3 MU of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount about 500 mg of drug per dose of pirfenidone or a specific pirfenidone analog orally qd, optionally in two or more divided doses per day, for the desired treatment duration.

In another embodiment, the invention provides a method using a synergistically effective amount of PEGASYS®PEGylated IFN-α2a and pirfenidone or a specific pirfenidone analog in the treatment of HCV infection in a patient comprising administering to the patient a dosage of PEGASYS® containing an amount of about 90 μg to about 360 μg of drug per dose of PEGASYS®, subcutaneously qw, qow, three times per month, or monthly, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In another embodiment, the invention provides a method using a synergistically effective amount of PEGASYS®PEGylated IFN-α2a and pirfenidone or a specific pirfenidone analog in the treatment of HCV infection in a patient comprising administering to the patient a dosage of PEGASYS® containing an amount of about 180 μg of drug per dose of PEGASYS®, subcutaneously qw, qow, three times per month, or monthly, in combination with a dosage of pirfenidone or a specific pirfenidone analog containing an amount of about 500 mg of drug per dose of pirfenidone or a specific pirfenidone analog orally qd, optionally in two or more divided doses per day, for the desired treatment duration.

In another embodiment, the invention provides a method using a synergistically effective amount of PEG-INTRON®PEGylated IFN-α2b and pirfenidone or a specific pirfenidone analog in the treatment of HCV infection in a patient comprising administering to the patient a dosage of PEG-INTRON® containing an amount of about 0.75 μg to about 3.0 μg of drug per kilogram of body weight per dose of PEG-INTRON®, subcutaneously qw, qow, three times per month, or monthly, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per month, for the desired treatment duration.

In another embodiment, the invention provides a method using a synergistically effective amount of PEG-INTRON®PEGylated IFN-α2b and pirfenidone or a specific pirfenidone analog in the treatment of HCV infection in a patient comprising administering to the patient a dosage of PEG-INTRON® containing an amount of about 1.5 μg of drug per kilogram of body weight per dose of PEG-INTRON®, subcutaneously qw, qow, three times per month, or monthly, in combination with a dosage of pirfenidone or a specific pirfenidone analog containing an amount of about 500 mg of drug per dose of pirfenidone or a specific pirfenidone analog orally qd, optionally in two or more divided doses per month, for the desired treatment duration.

High Dose Interferon Receptor Agonist in Combination with Pirfenidone

In addition to synergistic combinations of an interferon receptor agonist and pirfenidone or a pirfenidone analog, combination therapy involving administering a high dose of the interferon receptor agonist and an effective amount of pirfenidone or a pirfenidone analog for the treatment of an HCV infection is provided. Pirfenidone can reduce undesirable side effects of the interferon receptor agonist, thus permitting the use of higher doses. In some of these embodiments, combination therapy involves administering to an individual in need thereof a high dose of an IFN-α and pirfenidone or a pirfenidone analog for the treatment of an HCV infection.

In some of these embodiments, the interferon receptor agonist is administered at or near, or even exceeding the maximum tolerated dose (MTD). In this context, the term “MTD” refers to the maximum amount of the interferon receptor agonist tolerated by the patient in interferon receptor agonist monotherapy.

In another embodiment, the invention provides a method using an effective amount of INFERGEN®consensus IFN-α and pirfenidone or a specific pirfenidone analog in the treatment of HCV infection in a patient comprising administering to the patient a dosage of INFERGEN® containing an amount of about 5 μg to about 150 μg, of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 10,000 mg of drug per dose of pirfenidone or a specific pirfenidone analog orally qd, optionally in two or more divided doses per day, for the desired treatment duration.

In another embodiment, the invention provides a method using an effective amount of INFERGEN®consensus IFN-α and pirfenidone or a specific pirfenidone analog in the treatment of HCV infection in a patient comprising administering to the patient a dosage of INFERGEN® containing an amount of about 5 μg to about 45 μg, of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 3,000 mg of drug per dose of pirfenidone or a specific pirfenidone analog orally qd, optionally in two or more divided doses per day, for the desired treatment duration.

In another embodiment, the invention provides a method using an effective amount of INFERGEN®consensus IFN-α and pirfenidone or a specific pirfenidone analog in the treatment of HCV infection in a patient comprising administering to the patient a dosage of INFERGEN® containing an amount of about 45 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose of pirfenidone or a specific pirfenidone analog orally qd, optionally in two or more divided doses per day, for the desired treatment duration.

In another embodiment, the invention provides a method using an effective amount of a consensus IFN-α and pirfenidone or a specific pirfenidone analog in the treatment of HCV infection in a patient comprising administering to the patient a dosage of PEGylated consensus IFN-α (PEG-CIFN) containing an amount of about 50 μg to about 750 μg of CIFN amino acid weight per dose of PEG-CIFN, subcutaneously qw, qow, three times per month, or monthly, in combination with a dosage of pirfenidone or a specific pirfenidone analog containing an amount of about 1,000 mg to about 10,000 mg of drug per dose of pirfenidone or a specific pirfenidone analog orally qd, optionally in two or more divided doses per day, for the desired treatment duration.

In another embodiment, the invention provides a method using an effective amount of a consensus IFN-α and pirfenidone or a specific pirfenidone analog in the treatment of HCV infection in a patient comprising administering to the patient a dosage of PEGylated consensus IFN-α (PEG-CIFN) containing an amount of about 225 μg to about 300 μg of CIFN amino acid weight per dose of PEG-CIFN, subcutaneously qw, qow, three times per month, or monthly, in combination with a dosage of pirfenidone or a specific pirfenidone analog containing an amount of about 1,000 mg to about 3,000 mg of drug per dose of pirfenidone or a specific pirfenidone analog orally qd, optionally in two or more divided doses per day, for the desired treatment duration.

In another embodiment, the invention provides a method using an effective amount of a consensus IFN-α and pirfenidone or a specific pirfenidone analog in the treatment of HCV infection in a patient comprising administering to the patient a dosage of PEGylated consensus IFN-α (PEG-CIFN) containing an amount of about 225 μg to about 300 μg of CIFN amino acid weight per dose of PEG-CIFN, subcutaneously qw, qow, three times per month, or monthly, in combination with a dosage of pirfenidone or a specific pirfenidone analog containing an amount of about 1,000 mg to about 2,000 mg of drug per dose of pirfenidone or a specific pirfenidone analog orally qd, optionally in two or more divided doses per day, for the desired treatment duration.

In another embodiment, the invention provides a method using an effective amount of IFN-α 2a or 2b or 2c and pirfenidone or a specific pirfenidone analog in the treatment of HCV infection in a patient comprising administering to the patient a dosage of IFN-α 2a or 2b or 2c containing an amount of about 5 MU to about 100 MU of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount of about 1,000 mg to about 10,000 mg of drug per dose of pirfenidone or a specific pirfenidone analog orally qd, optionally in two or more divided doses per day, for the desired treatment duration.

In another embodiment, the invention provides a method using an effective amount of IFN-α 2a or 2b or 2c and pirfenidone or a specific pirfenidone analog in the treatment of HCV infection in a patient comprising administering to the patient a dosage of IFN-α 2a or 2b or 2c containing an amount of about 15 MU to about 50 MU of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount about 1,000 of mg to about 3,000 mg of drug per dose of pirfenidone or a specific pirfenidone analog orally qd, optionally in two or more divided doses per day, for the desired treatment duration.

In another embodiment, the invention provides a method using an effective amount of IFN-α 2a or 2b or 2c and pirfenidone or a specific pirfenidone analog in the treatment of HCV infection in a patient comprising administering to the patient a dosage of IFN-α 2a or 2b or 2c containing an amount of about 15 MU of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount about 1,000 mg to about 2,000 mg of drug per dose of pirfenidone or a specific pirfenidone analog orally qd, optionally in two or more divided doses per day, for the desired treatment duration.

In another embodiment, the invention provides a method using an effective amount of PEGASYS®PEGylated IFN-α2a and pirfenidone or a specific pirfenidone analog in the treatment of HCV infection in a patient comprising administering to the patient a dosage of PEGASYS® containing an amount of about 450 μg to about 1800 μg of drug per dose of PEGASYS®, subcutaneously qw, qow, three times per month, or monthly, in combination with a dosage of pirfenidone or a specific pirfenidone analog containing an amount of about 1,000 mg to about 10,000 mg of drug per dose of pirfenidone or a specific pirfenidone analog orally qd, optionally in two or more divided doses per day, for the desired treatment duration.

In another embodiment, the invention provides a method using an effective amount of PEGASYS®PEGylated IFN-α2a and pirfenidone or a specific pirfenidone analog in the treatment of HCV infection in a patient comprising administering to the patient a dosage of PEGASYS® containing an amount of about 900 μg of drug per dose of PEGASYS®, subcutaneously qw, qow, three times per month, or monthly, in combination with a dosage of pirfenidone or a specific pirfenidone analog containing an amount of about 1,000 mg to about 2,000 mg of drug per dose of pirfenidone or a specific pirfenidone analog orally qd, optionally in two or more divided doses per day, for the desired treatment duration.

In another embodiment, the invention provides a method using an effective amount of PEG-INTRON®PEGylated IFN-α2b and pirfenidone or a specific pirfenidone analog in the treatment of HCV infection in a patient comprising administering to the patient a dosage of PEG-INTRON® containing an amount of about 0.375 μg to about 15.0 μg of drug per kilogram of body weight per dose of PEG-INTRON®, subcutaneously qw, qow, three times per month, or monthly, in combination with a dosage of pirfenidone or a specific pirfenidone analog containing an amount of about 1,000 mg to about 10,000 mg of drug per dose of pirfenidone or a specific pirfenidone analog orally qd, optionally in two or more divided doses per month, for the desired treatment duration.

In another embodiment, the invention provides a method using an effective amount of PEG-INTRON®PEGylated IFN-α2b and pirfenidone or a specific pirfenidone analog in the treatment of HCV infection in a patient comprising administering to the patient a dosage of PEG-INTRON® containing an amount of about 7.5 μg of drug per kilogram of body weight per dose of PEG-INTRON®, subcutaneously qw, qow, three times per month, or monthly, in combination with a dosage of pirfenidone or a specific pirfenidone analog containing an amount of about 1,000 mg to about 2,000 mg of drug per dose of pirfenidone or a specific pirfenidone analog orally qd, optionally in two or more divided doses per month, for the desired treatment duration.

Combination Therapy with Ribavirin

The invention also provides methods for the treatment of an HCV infection in which ribavirin therapy is added to any of the interferon receptor agonist and pirfenidone or a pirfenidone analog combination therapies described above. In some embodiments, the interferon receptor agonist and pirfenidone or a pirfenidone analog combination therapy is modified to include a ribavirin regimen of 800 mg to 1200 mg ribavirin orally qd for the specified duration of therapy. In other embodiments, the interferon receptor agonist and pirfenidone or a pirfenidone analog combination therapy is modified to include a ribavirin regimen of 1000 mg ribavirin orally qd for the specified duration of therapy. In additional embodiments, the interferon receptor agonist and pirfenidone or a pirfenidone analog combination therapy is modified to include a ribavirin regimen of about 10 mg of ribavirin/kg body weight orally qd for the specified duration of therapy. The daily ribavirin dosage can be administered in one dose per day or in divided doses, including one, two, three or four doses, per day.

3. Treatment of West Nile Viral Injection

The present invention provides methods of treating West Nile viral infection by administering a therapeutically effective amount of an interferon receptor agonist and pirfenidone or a pirfenidone analog to an individual in need thereof. Individuals who are to be treated according to the methods of the invention include individuals who have been clinically diagnosed with West Nile viral infection, as well as individuals who exhibit one or more of the signs and symptoms of clinical infection but have not yet been diagnosed with West Nile viral infection.

In carrying out combination therapy with an interferon receptor agonist and pirfenidone (or a pirfenidone analog) for West Nile virus infection, effective amounts of the interferon receptor agonist and pirfenidone or a pirfenidone analog are administered. In some embodiments, a low dose of interferon receptor agonist is administered in synergistic combination with pirfenidone or a pirfenidone analog, as described above. In other embodiments, a high dose of interferon receptor agonist is administered in combination with pirfenidone or a pirfenidone analog, as described above.

Effective dosages of IFN-α generally range from about 3 μg/dose to about 135 μg/dose. Effective dosages of Infergen®consensusIFN-α can contain an amount of about 3 μg, about 9 μg, about 15 μg, about 18 μg, or about 27 μg of drug per dose. Effective dosages of IFN-α2a and IFN-α2b can contain an amount of about 3 million Units (MU) to about 10 MU of drug per dose. Effective dosages of PEGASYS®PEGylated IFN-α2a contain an amount of about 90 μg to about 180 μg, or about 135 μg, of drug per dose. Effective dosages of PEG-INTRON®PEGylated IFN-α2b can contain an amount of about 0.5 μg to about 1.5 μg of drug per kg of body weight per dose. Effective dosages of PEGylated consensus interferon (PEG-CIFN) can contain an amount of about 18 μg to about 90 μg, or about 27 μg to about 60 μg, or about 45 μg, of CIFN amino acid weight per dose of PEG-CIFN.

Where the interferon receptor agonist is an IFN-β, in general, effective dosages of IFN-β can range from 3 μg to about 300 μg. Exemplary effective dosages of an IFN-β are 30 μg, 44 μg, and 300 μg.

Where the interferon receptor agonist is an IFN-γ, suitable dosages of IFN-γ can range from about 25 μg/dose to about 300 μg/dose, or about 100 μg/dose to about 1,000 μg/dose.

In many embodiments, interferon receptor agonist and/or pirfenidone or pirfenidone analog is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. Dosage regimens can include tid, bid, qd, qod, biw, tiw, qw, qow, three times per month, or monthly administrations.

In many embodiments, multiple doses of an interferon receptor agonist are administered. For example, the interferon receptor agonist is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid) over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Effective dosages of pirfenidone or specific pirfenidone analogs can range from about 5 mg/kg/day to about 125 mg/kg/day, or at a fixed dosage of about 400 mg to about 3600 mg per day, administered orally. Other doses and formulations of pirfenidone and specific pirfenidone analogs suitable for use in the treatment of an alphavirus infection are described in U.S. Pat. Nos. 3,974,281; 3,839,346; 4,042,699; 4,052,509; 5,310,562; 5,518,729; 5,716,632; and 6,090,822.

Those of skill in the art will readily appreciate that dose levels of pirfenidone or pirfenidone analog can vary as a function of the specific compound, the severity of the symptoms and the susceptibility of the subject to side effects. Preferred dosages for a given compound are readily determinable by those of skill in the art by a variety of means.

Pirfenidone (or a pirfenidone analog) can be administered once per month, twice per month, three times per month, every other week, once per week, twice per week, three times per week, four times per week, five times per week, six times per week, every other day, daily, twice a day, or three times a day, or in divided daily doses ranging from once daily to 5 times daily over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

An interferon receptor agonist and pirfenidone (or pirfenidone analog) are generally administered in separate formulations. An interferon receptor agonist and pirfenidone (or pirfenidone analog) may be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

Low Dose Interferon Receptor Agonist in Synergistic Combination with Pirfenidone

In some embodiments, the invention provides methods using a synergistically effective amount of an interferon receptor agonist and pirfenidone or a pirfenidone analog in the treatment of West Nile viral (WNV) infection in a patient. In these embodiments, a low dose of an interferon receptor agonist is administered in combination therapy with pirfenidone or a pirfenidone analog. In some embodiments, the invention provides a method using a synergistically effective amount of an IFN-α and pirfenidone or pirfenidone analog in the treatment of the WNV infection in a patient in need thereof. In one embodiment, the invention provides a method using a synergistically effective amount of a consensus IFN-α and pirfenidone or a pirfenidone analog in the treatment of WNV infection in a patient in need thereof.

In another embodiment, the invention provides a method using a synergistically effective amount of INFERGEN®consensus IFN-α and pirfenidone or a specific pirfenidone analog in the treatment of WNV infection in a patient comprising administering to the patient a dosage of INFERGEN® containing an amount of about 1 μg to about 30 μg, of drug per dose of INFERGEN®, subcutaneously qd, qod, tiw, or biw, or per day substantially continuously or continuously, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In another embodiment, the invention provides a method using a synergistically effective amount of INFERGEN®consensus IFN-α and pirfenidone or a specific pirfenidone analog in the treatment of WNV infection in a patient comprising administering to the patient a dosage of INFERGEN® containing an amount of about 1 μg to about 9 μg, of drug per dose of INFERGEN®, subcutaneously qd, qod, tiw, or biw, or per day substantially continuously or continuously, in combination with a dosage of pirfenidone or a specific pirfenidone analog containing an amount of about 100 mg to about 1,000 mg of drug per dose of pirfenidone or a specific pirfenidone analog orally qd, optionally in two or more divided doses per day, for the desired treatment duration.

In another embodiment, the invention provides a method using a synergistically effective amount of INFERGEN®consensus IFN-α and pirfenidone or a specific pirfenidone analog in the treatment of WNV infection in a patient comprising administering to the patient a dosage of INFERGEN® containing an amount of about 9 μg, of drug per dose of INFERGEN®, subcutaneously qd, qod, tiw, or biw, or per day substantially continuously or continuously, in combination with a dosage of pirfenidone or a specific pirfenidone analog containing an amount of about 500 mg of drug per dose of pirfenidone or a specific pirfenidone analog orally qd, optionally in two or more divided doses per day, for the desired treatment duration.

In another embodiment, the invention provides a method using a synergistically effective amount of a consensus IFN-α and pirfenidone or a specific pirfenidone analog in the treatment of WNV infection in a patient comprising administering to the patient a dosage of PEGylated consensus IFN-α (PEG-CIFN) containing an amount of about 10 μg to about 150 μg of CIFN amino acid weight per dose of PEG-CIFN, subcutaneously qw, qow, three times per month, or monthly, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In another embodiment, the invention provides a method using a synergistically effective amount of a consensus IFN-α and pirfenidone or a specific pirfenidone analog in the treatment of WNV infection in a patient comprising administering to the patient a dosage of PEGylated consensus IFN-α (PEG-CIFN) containing an amount of about 45 μg to about 60 μg of CIFN amino acid weight per dose of PEG-CIFN, subcutaneously qw, qow, three times per month, or monthly, in combination with a dosage of pirfenidone or a specific pirfenidone analog containing an amount of about 10 mg to about 1,000 mg of drug per dose of pirfenidone or a specific pirfenidone analog orally qd, optionally in two or more divided doses per day, for the desired treatment duration.

In another embodiment, the invention provides a method using a synergistically effective amount of a consensus IFN-α and pirfenidone or a specific pirfenidone analog in the treatment of WNV infection in a patient comprising administering to the patient a dosage of PEGylated consensus IFN-α (PEG-CIFN) containing an amount of about 45 μg to about 60 μg of CIFN amino acid weight per dose of PEG-CIFN, subcutaneously qw, qow, three times per month, or monthly, in combination with a dosage of pirfenidone or a specific pirfenidone analog containing an amount of about 500 mg of drug per dose of pirfenidone or a specific pirfenidone analog orally qd, optionally in two or more divided doses per day, for the desired treatment duration.

In another embodiment, the invention provides a method using a synergistically effective amount of IFN-α 2a or 2b or 2c and pirfenidone or a specific pirfenidone analog in the treatment of WNV infection in a patient comprising administering to the patient a dosage of IFN-α 2a or 2b or 2c containing an amount of about 1 MU to about 20 MU of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In another embodiment, the invention provides a method using a synergistically effective amount of IFN-α 2a or 2b or 2c and pirfenidone or a specific pirfenidone analog in the treatment of WNV infection in a patient comprising administering to the patient a dosage of IFN-α 2a or 2b or 2c containing an amount of about 3 MU to about 10 MU of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount about 100 of mg to about 1,000 mg of drug per dose of pirfenidone or a specific pirfenidone analog orally qd, optionally in two or more divided doses per day, for the desired treatment duration.

In another embodiment, the invention provides a method using a synergistically effective amount of IFN-α 2a or 2b or 2c and pirfenidone or a specific pirfenidone analog in the treatment of WNV infection in a patient comprising administering to the patient a dosage of IFN-α 2a or 2b or 2c containing an amount of about 3 MU of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount about 500 mg of drug per dose of pirfenidone or a specific pirfenidone analog orally qd, optionally in two or more divided doses per day, for the desired treatment duration.

In another embodiment, the invention provides a method using a synergistically effective amount of PEGASYS®PEGylated IFN-α2a and pirfenidone or a specific pirfenidone analog in the treatment of WNV infection in a patient comprising administering to the patient a dosage of PEGASYS® containing an amount of about 90 μg to about 360 μg of drug per dose of PEGASYS®, subcutaneously qw, qow, three times per month, or monthly, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In another embodiment, the invention provides a method using a synergistically effective amount of PEGASYS®PEGylated IFN-α2a and pirfenidone or a specific pirfenidone analog in the treatment of WNV infection in a patient comprising administering to the patient a dosage of PEGASYS® containing an amount of about 180 μg of drug per dose of PEGASYS®, subcutaneously qw, qow, three times per month, or monthly, in combination with a dosage of pirfenidone or a specific pirfenidone analog containing an amount of about 500 mg of drug per dose of pirfenidone or a specific pirfenidone analog orally qd, optionally in two or more divided doses per day, for the desired treatment duration.

In another embodiment, the invention provides a method using a synergistically effective amount of PEG-INTRON®PEGylated IFN-α2b and pirfenidone or a specific pirfenidone analog in the treatment of WNV infection in a patient comprising administering to the patient a dosage of PEG-INTRON® containing an amount of about 0.75 μg to about 3.0 μg of drug per kilogram of body weight per dose of PEG-INTRON®, subcutaneously qw, qow, three times per month, or monthly, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per month, for the desired treatment duration.

In another embodiment, the invention provides a method using a synergistically effective amount of PEG-INTRON®PEGylated IFN-α2b and pirfenidone or a specific pirfenidone analog in the treatment of WNV infection in a patient comprising administering to the patient a dosage of PEG-INTRON® containing an amount of about 1.5 μg of drug per kilogram of body weight per dose of PEG-INTRON®, subcutaneously qw, qow, three times per month, or monthly, in combination with a dosage of pirfenidone or a specific pirfenidone analog containing an amount of about 500 mg of drug per dose of pirfenidone or a specific pirfenidone analog orally qd, optionally in two or more divided doses per month, for the desired treatment duration.

High Dose Interferon Receptor Agonist in Combination with Pirfenidone

In addition to synergistic combinations of an interferon receptor agonist and pirfenidone or a pirfenidone analog, combination therapy involving administering a high dose of an interferon receptor agonist and an effective amount of pirfenidone or a pirfenidone analog is provided. Pirfenidone can reduce undesirable side effects of interferon receptor agonist, thus permitting the use of higher doses.

In some of these embodiments, the interferon receptor agonist is administered at or near, or even exceeding the maximum tolerated dose (MTD). In this context, the term “MTD” refers to the maximum amount of the interferon receptor agonist tolerated by the patient in interferon receptor agonist monotherapy.

In another embodiment, the invention provides a method using an effective amount of INFERGEN®consensus IFN-α and pirfenidone or a specific pirfenidone analog in the treatment of WNV infection in a patient comprising administering to the patient a dosage of INFERGEN® containing an amount of about 5 μg to about 150 μg, of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 10,000 mg of drug per dose of pirfenidone or a specific pirfenidone analog orally qd, optionally in two or more divided doses per day, for the desired treatment duration.

In another embodiment, the invention provides a method using an effective amount of INFERGEN®consensus IFN-α and pirfenidone or a specific pirfenidone analog in the treatment of WNV infection in a patient comprising administering to the patient a dosage of INFERGEN® containing an amount of about 5 μg to about 45 μg, of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 3,000 mg of drug per dose of pirfenidone or a specific pirfenidone analog orally qd, optionally in two or more divided doses per day, for the desired treatment duration.

In another embodiment, the invention provides a method using an effective amount of INFERGEN®consensus IFN-α and pirfenidone or a specific pirfenidone analog in the treatment of WNV infection in a patient comprising administering to the patient a dosage of INFERGEN® containing an amount of about 45 μg of drug per dose of INFERGEN®, subcutaneously qd, qod, tiw, or biw, or per day substantially continuously or continuously, in combination with a dosage of pirfenidone or a specific pirfenidone analog containing an amount of about 1,000 mg to about 2,000 mg of drug per dose of pirfenidone or a specific pirfenidone analog orally qd, optionally in two or more divided doses per day, for the desired treatment duration.

In another embodiment, the invention provides a method using an effective amount of a consensus IFN-α and pirfenidone or a specific pirfenidone analog in the treatment of WNV infection in a patient comprising administering to the patient a dosage of PEGylated consensus IFN-α (PEG-CIFN) containing an amount of about 50 μg to about 750 μg of CIFN amino acid weight per dose of PEG-CIFN, subcutaneously qw, qow, three times per month, or monthly, in combination with a dosage of pirfenidone or a specific pirfenidone analog containing an amount of about 1,000 mg to about 10,000 mg of drug per dose of pirfenidone or a specific pirfenidone analog orally qd, optionally in two or more divided doses per day, for the desired treatment duration.

In another embodiment, the invention provides a method using an effective amount of a consensus IFN-α and pirfenidone or a specific pirfenidone analog in the treatment of WNV infection in a patient comprising administering to the patient a dosage of PEGylated consensus IFN-α (PEG-CIFN) containing an amount of about 225 μg to about 300 μg of CIFN amino acid weight per dose of PEG-CIFN, subcutaneously qw, qow, three times per month, or monthly, in combination with a dosage of pirfenidone or a specific pirfenidone analog containing an amount of about 1,000 mg to about 3,000 mg of drug per dose of pirfenidone or a specific pirfenidone analog orally qd, optionally in two or more divided doses per day, for the desired treatment duration.

In another embodiment, the invention provides a method using an effective amount of a consensus IFN-α and pirfenidone or a specific pirfenidone analog in the treatment of WNV infection in a patient comprising administering to the patient a dosage of PEGylated consensus IFN-α (PEG-CIFN) containing an amount of about 225 μg to about 300 μg of CIFN amino acid weight per dose of PEG-CIFN, subcutaneously qw, qow, three times per month, or monthly, in combination with a dosage of pirfenidone or a specific pirfenidone analog containing an amount of about 1,000 mg to about 2,000 mg of drug per dose of pirfenidone or a specific pirfenidone analog orally qd, optionally in two or more divided doses per day, for the desired treatment duration.

In another embodiment, the invention provides a method using an effective amount of IFN-α 2a or 2b or 2c and pirfenidone or a specific pirfenidone analog in the treatment of WNV infection in a patient comprising administering to the patient a dosage of IFN-α 2a or 2b or 2c containing an amount of about 5 MU to about 100 MU of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount of about 1,000 mg to about 10,000 mg of drug per dose of pirfenidone or a specific pirfenidone analog orally qd, optionally in two or more divided doses per day, for the desired treatment duration.

In another embodiment, the invention provides a method using an effective amount of IFN-α 2a or 2b or 2c and pirfenidone or a specific pirfenidone analog in the treatment of WNV infection in a patient comprising administering to the patient a dosage of IFN-α 2a or 2b or 2c containing an amount of about 15 MU to about 50 MU of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount about 1,000 of mg to about 3,000 mg of drug per dose of pirfenidone or a specific pirfenidone analog orally qd, optionally in two or more divided doses per day, for the desired treatment duration.

In another embodiment, the invention provides a method using an effective amount of IFN-α 2a or 2b or 2c and pirfenidone or a specific pirfenidone analog in the treatment of WNV infection in a patient comprising administering to the patient a dosage of IFN-α 2a or 2b or 2c containing an amount of about 15 MU of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount about 1,000 mg to about 2,000 mg of drug per dose of pirfenidone or a specific pirfenidone analog orally qd, optionally in two or more divided doses per day, for the desired treatment duration.

In another embodiment, the invention provides a method using an effective amount of PEGASYS®PEGylated IFN-α2a and pirfenidone or a specific pirfenidone analog in the treatment of WNV infection in a patient comprising administering to the patient a dosage of PEGASYS® containing an amount of about 450 μg to about 1800 μg of drug per dose of PEGASYS®, subcutaneously qw, qow, three times per month, or monthly, in combination with a dosage of pirfenidone or a specific pirfenidone analog containing an amount of about 1,000 mg to about 10,000 mg of drug per dose of pirfenidone or a specific pirfenidone analog orally qd, optionally in two or more divided doses per day, for the desired treatment duration.

In another embodiment, the invention provides a method using an effective amount of PEGASYS®PEGylated IFN-α2a and pirfenidone or a specific pirfenidone analog in the treatment of WNV infection in a patient comprising administering to the patient a dosage of PEGASYS® containing an amount of about 900 μg of drug per dose of PEGASYS®, subcutaneously qw, qow, three times per month, or monthly, in combination with a dosage of pirfenidone or a specific pirfenidone analog containing an amount of about 1,000 mg to about 2,000 mg of drug per dose of pirfenidone or a specific pirfenidone analog orally qd, optionally in two or more divided doses per day, for the desired treatment duration.

In another embodiment, the invention provides a method using an effective amount of PEG-INTRON®PEGylated IFN-α2b and pirfenidone or a specific pirfenidone analog in the treatment of WNV infection in a patient comprising administering to the patient a dosage of PEG-INTRON® containing an amount of about 0.375 μg to about 15.0 μg of drug per kilogram of body weight per dose of PEG-INTRON®, subcutaneously qw, qow, three times per month, or monthly, in combination with a dosage of pirfenidone or a specific pirfenidone analog containing an amount of about 1,000 mg to about 10,000 mg of drug per dose of pirfenidone or a specific pirfenidone analog orally qd, optionally in two or more divided doses per month, for the desired treatment duration.

In another embodiment, the invention provides a method using an effective amount of PEG-INTRON®PEGylated IFN-α2b and pirfenidone or a specific pirfenidone analog in the treatment of WNV infection in a patient comprising administering to the patient a dosage of PEG-INTRON® containing an amount of about 7.5 μg of drug per kilogram of body weight per dose of PEG-INTRON®, subcutaneously qw, qow, three times per month, or monthly, in combination with a dosage of pirfenidone or a specific pirfenidone analog containing an amount of about 1,000 mg to about 2,000 mg of drug per dose of pirfenidone or a specific pirfenidone analog orally qd, optionally in two or more divided doses per month, for the desired treatment duration.

Combination Therapy with Ribavirin

The invention also provides methods for the treatment of WNV infection in which ribavirin therapy is added to any of the interferon receptor agonist and pirfenidone or a pirfenidone analog combination therapies described above. In some embodiments, the interferon receptor agonist and pirfenidone or a pirfenidone analog combination therapy is modified to include a ribavirin regimen of 800 mg to 1200 mg ribavirin orally qd for the specified duration of therapy. In other embodiments, the interferon receptor agonist and pirfenidone or a pirfenidone analog combination therapy is modified to include a ribavirin regimen of 1000 mg ribavirin orally qd for the specified duration of therapy. In additional embodiments, the interferon receptor agonist and pirfenidone or a pirfenidone analog combination therapy is modified to include a ribavirin regimen of about 10 mg of ribavirin/kg body weight orally qd for the specified duration of therapy. The daily ribavirin dosage can be administered in one dose per day or in divided doses, including one, two, three or four doses, per day.

4. Treatment of Liver Fibrosis

Individuals with liver fibrosis who are suitable for treatment according to the methods of the invention include individuals who have been clinically diagnosed with liver fibrosis, as well as individuals who have not yet developed clinical liver fibrosis but who are considered at risk of developing liver fibrosis. Such individuals include, but are not limited to, individuals who are infected with HCV; individuals who are infected with HBV; individuals who are infected with Schistosoma mansoni; individuals who have been exposed to chemical agents known to result in liver fibrosis; individuals who have been diagnosed with Wilson's disease; individuals diagnosed with hemochromatosis; and individuals with alcoholic liver disease; individuals with non-alcoholic steatohepatitis; individuals with autoimmune hepatitis; individuals with primary sclerosing cholangitis, primary biliary cirrhosis, or alpha-1-antitrysin deficiency.

In one aspect, the invention provides a method of treating liver fibrosis in a patient comprising administering to the patient an amount of an interferon receptor agonist and pirfenidone or a pirfenidone analog to reduce liver fibrosis.

In another aspect, the invention provides a method of increasing liver function in a patient suffering from liver fibrosis, comprising administering to the patient an amount of an interferon receptor agonist and pirfenidone or a pirfenidone analog effective to increase liver function.

In another aspect, the invention provides a method of reducing the incidence of a complication of cirrhosis of the liver in a patient suffering from liver fibrosis, comprising administering to the patient an amount of interferon receptor agonist and pirfenidone or a pirfenidone analog effective to reduce the incidence of a complication of cirrhosis of the liver.

Effective dosages of IFN-α generally range from about 3 μg/dose to about 135 μg/dose. In one embodiment, the methods of the invention for the treatment of liver fibrosis described above can be carried out by administering to the patient a dosage of INFERGEN®consensus IFN-α containing an amount of about 3 μg, about 9 μg, about 15 μg, about 18 μg, or about 27 μg, of drug per dose of INFERGEN®, subcutaneously qd, qod, tiw, biw, qw, qow, three times per month, once monthly, or per day substantially continuously or continuously, and a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α therapy.

In another embodiment, the methods of the invention for treatment of liver fibrosis described above can be carried out by administering to the patient a dosage of IFN-α2a or IFN-α2b containing an amount of about 3 million Units (MU to about 10 MU of drug per dose of IFN-α2a or IFN-α2b, subcutaneously qd, qod, tiw, biw, qw, qow, three times per month, once monthly, or per day substantially continuously or continuously, and a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α therapy.

In another embodiment, the methods of the invention for treatment of liver fibrosis described above can be carried out by administering to the patient a dosage of PEGASYS®PEGylated IFN-α2a containing an amount of about 90 μg to about 180 μg, or about 135 μg, of drug per dose of PEGASYS®, subcutaneously qw qow, three times per month, or monthly and a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α.

In another embodiment, the methods of the invention for treatment of liver fibrosis described above can be carried out by administering to the patient a dosage of PEG-INTRON®PEGylated IFN-α2b containing an amount of about 0.5 μg to about 1.5 μg of drug per kg body weight per dose of PEG-INTRON®, subcutaneously qw, qow, three times per month, or monthly and a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α therapy.

In another embodiment, the methods of the invention for treatment of liver fibrosis described above can be carried out by administering to the patient a dosage of PEGylated consensus interferon (PEG-CIFN) containing an amount of about 18 μg to about 90 μg, or about 27 μg to about 60 μg, or about 45 μg, of CIFN amino acid weight per dose of PEG-CIFN, subcutaneously qw, qow, three times per month, or monthly and a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α and IFN-γ therapy.

Where the interferon receptor agonist is an IFN-β, in general, effective dosages of IFN-β can range from 3 μg to about 300 μg. Exemplary effective dosages of an IFN-β are 30 μg, 44 μg, and 300 μg.

Where the interferon receptor agonist is an IFN-γ, suitable dosages of IFN-γ range from about 25 μg/dose to about 300 μg/dose.

In many embodiments, an interferon analog and pirfenidone or a pirfenidone analog is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time.

Low Dose Interferon Receptor Agonist in Synergistic Combination with Pirfenidone

In some embodiments, the invention provides methods using a synergistically effective amount of an interferon receptor agonist and pirfenidone or a pirfenidone analog in the treatment of liver fibrosis in a patient. In these embodiments, a low dose of an interferon receptor agonist is administered in combination therapy with pirfenidone or a pirfenidone analog. In some embodiments, the invention provides a method using a synergistically effective amount of an IFN-α and pirfenidone or pirfenidone analog in the treatment of the liver fibrosis in a patient in need thereof. In one embodiment, the invention provides a method using a synergistically effective amount of a consensus IFN-α and pirfenidone or a pirfenidone analog in the treatment of liver fibrosis in a patient in need thereof.

In another embodiment, the invention provides a method using a synergistically effective amount of INFERGEN®consensus IFN-α and pirfenidone or a specific pirfenidone analog in the treatment of liver fibrosis in a patient comprising administering to the patient a dosage of INFERGEN® containing an amount of about 1 μg to about 30 μg, of drug per dose of INFERGEN®, subcutaneously qd, qod, tiw, or biw, or per day substantially continuously or continuously, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In another embodiment, the invention provides a method using a synergistically effective amount of INFERGEN®consensus IFN-α and pirfenidone or a specific pirfenidone analog in the treatment of liver fibrosis in a patient comprising administering to the patient a dosage of INFERGEN® containing an amount of about 1 μg to about 9 μg of drug per dose of INFERGEN®, subcutaneously qd, qod, tiw, or biw, or per day substantially continuously or continuously, in combination with a dosage of pirfenidone or a specific pirfenidone analog containing an amount of about 100 mg to about 1,000 mg of drug per dose of pirfenidone or a specific pirfenidone analog orally qd, optionally in two or more divided doses per day, for the desired treatment duration.

In another embodiment, the invention provides a method using a synergistically effective amount of INFERGEN®consensus IFN-α and pirfenidone or a specific pirfenidone analog in the treatment of liver fibrosis in a patient comprising administering to the patient a dosage of INFERGEN® containing an amount of about 9 μg of drug per dose of INFERGEN®, subcutaneously qd, qod, tiw, or biw, or per day substantially continuously or continuously, in combination with a dosage of pirfenidone or a specific pirfenidone analog containing an amount of about 500 mg of drug per dose of pirfenidone or a specific pirfenidone analog orally qd, optionally in two or more divided doses per day, for the desired treatment duration.

In another embodiment, the invention provides a method using a synergistically effective amount of a consensus IFN-α and pirfenidone or a specific pirfenidone analog in the treatment of liver fibrosis in a patient comprising administering to the patient a dosage of PEGylated consensus IFN-α (PEG-CIFN) containing an amount of about 10 μg to about 150 μg of CIFN amino acid weight per dose of PEG-CIFN, subcutaneously qw, qow, three times per month, or monthly, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In another embodiment, the invention provides a method using a synergistically effective amount of a consensus IFN-α and pirfenidone or a specific pirfenidone analog in the treatment of liver fibrosis in a patient comprising administering to the patient a dosage of PEGylated consensus IFN-α (PEG-CIFN) containing an amount of about 45 μg to about 60 μg of CIFN amino acid weight per dose of PEG-CIFN, subcutaneously qw, qow, three times per month, or monthly, in combination with a dosage of pirfenidone or a specific pirfenidone analog containing an amount of about 10 mg to about 1,000 mg of drug per dose of pirfenidone or a specific pirfenidone analog orally qd, optionally in two or more divided doses per day, for the desired treatment duration.

In another embodiment, the invention provides a method using a synergistically effective amount of a consensus IFN-α and pirfenidone or a specific pirfenidone analog in the treatment of liver fibrosis in a patient comprising administering to the patient a dosage of PEGylated consensus IFN-α (PEG-CIFN) containing an amount of about 45 μg to about 60 μg of CIFN amino acid weight per dose of PEG-CIFN, subcutaneously qw, qow, three times per month, or monthly, in combination with a dosage of pirfenidone or a specific pirfenidone analog containing an amount of about 500 mg of drug per dose of pirfenidone or a specific pirfenidone analog orally qd, optionally in two or more divided doses per day, for the desired treatment duration.

In another embodiment, the invention provides a method using a synergistically effective amount of IFN-α 2a or 2b or 2c and pirfenidone or a specific pirfenidone analog in the treatment of liver fibrosis in a patient comprising administering to the patient a dosage of IFN-α 2a or 2b or 2c containing an amount of about 1 MU to about 20 MU, of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In another embodiment, the invention provides a method using a synergistically effective amount of IFN-α 2a or 2b or 2c and pirfenidone or a specific pirfenidone analog in the treatment of liver fibrosis in a patient comprising administering to the patient a dosage of IFN-α 2a or 2b or 2c containing an amount of about 3 MU to about 10 MU of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount about 100 of mg to about 1,000 mg of drug per dose of pirfenidone or a specific pirfenidone analog orally qd, optionally in two or more divided doses per day, for the desired treatment duration.

In another embodiment, the invention provides a method using a synergistically effective amount of IFN-α 2a or 2b or 2c and pirfenidone or a specific pirfenidone analog in the treatment of liver fibrosis in a patient comprising administering to the patient a dosage of IFN-α 2a or 2b or 2c containing an amount of about 3 MU of drug per dose of IFN-α 2a or 2b or 2c, subcutaneously qd, qod, tiw, or biw, or per day substantially continuously or continuously, in combination with a dosage of pirfenidone or a specific pirfenidone analog containing an amount about 500 mg of drug per dose of pirfenidone or a specific pirfenidone analog orally qd, optionally in two or more divided doses per day, for the desired treatment duration.

In another embodiment, the invention provides a method using a synergistically effective amount of PEGASYS®PEGylated IFN-α2a and pirfenidone or a specific pirfenidone analog in the treatment of liver fibrosis in a patient comprising administering to the patient a dosage of PEGASYS® containing an amount of about 90 μg to about 360 μg of drug per dose of PEGASYS®, subcutaneously qw, qow, three times per month, or monthly, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In another embodiment, the invention provides a method using a synergistically effective amount of PEGASYS®PEGylated IFN-α2a and pirfenidone or a specific pirfenidone analog in the treatment of liver fibrosis in a patient comprising administering to the patient a dosage of PEGASYS® containing an amount of about 180 μg of drug per dose of PEGASYS®, subcutaneously qw, qow, three times per month, or monthly, in combination with a dosage of pirfenidone or a specific pirfenidone analog containing an amount of about 500 mg of drug per dose of pirfenidone or a specific pirfenidone analog orally qd, optionally in two or more divided doses per day, for the desired treatment duration.

In another embodiment, the invention provides a method using a synergistically effective amount of PEG-INTRON®PEGylated IFN-α2b and pirfenidone or a specific pirfenidone analog in the treatment of liver fibrosis in a patient comprising administering to the patient a dosage of PEG-INTRON® containing an amount of about 0.75 μg to about 3.0 μg of drug per kilogram of body weight per dose of PEG-INTRON®, subcutaneously qw, qow, three times per month, or monthly, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per month, for the desired treatment duration.

In another embodiment, the invention provides a method using a synergistically effective amount of PEG-INTRON®PEGylated IFN-α2b and pirfenidone or a specific pirfenidone analog in the treatment of liver fibrosis in a patient comprising administering to the patient a dosage of PEG-INTRON® containing an amount of about 1.5 μg of drug per kilogram of body weight per dose of PEG-INTRON®, subcutaneously qw, qow, three times per month, or monthly, in combination with a dosage of pirfenidone or a specific pirfenidone analog containing an amount of about 500 mg of drug per dose of pirfenidone or a specific pirfenidone analog orally qd, optionally in two or more divided doses per month, for the desired treatment duration.

Additional Therapeutic Agents

In some embodiments, the methods provide for combination therapy comprising administering an interferon receptor agonist, pirfenidone or a pirfenidone analog, and an additional therapeutic agent such as ribavirin. In addition, in some embodiments, the method provide for combination therapy comprising administering two different interferon receptor agonists, and pirfenidone or a pirfenidone analog.

Interferon Receptor Agonist, Pirfenidone or Pirfenidone Analog, and an Additional Therapeutic Agent

In some embodiments, the additional therapeutic agent(s) is administered during the entire course of interferon receptor agonist treatment, and the beginning and end of the treatment periods coincide. In other embodiments, the additional therapeutic agent(s) is administered for a period of time that is overlapping with that of the interferon receptor agonist/pirfenidone (or a pirfenidone analog) combination treatment, e.g., treatment with the additional therapeutic agent(s) begins before the interferon receptor agonist/pirfenidone (or a pirfenidone analog) combination treatment begins and ends before the interferon receptor agonist/pirfenidone (or a pirfenidone analog) combination treatment ends; treatment with the additional therapeutic agent(s) begins after the interferon receptor agonist/pirfenidone (or a pirfenidone analog) combination treatment begins and ends after the interferon receptor agonist/pirfenidone (or a pirfenidone analog) combination treatment ends; treatment with the additional therapeutic agent(s) begins after the interferon receptor agonist/pirfenidone (or a pirfenidone analog) combination treatment begins and ends before the interferon receptor agonist/pirfenidone (or a pirfenidone analog) combination treatment ends; or treatment with the additional therapeutic agent(s) begins before the interferon receptor agonist/pirfenidone (or a pirfenidone analog) combination treatment begins and ends after the interferon receptor agonist/pirfenidone (or a pirfenidone analog) combination treatment ends.

The interferon receptor agonist/pirfenidone (or a pirfenidone analog) combination therapy can be administered together with (i.e., simultaneously in separate formulations; simultaneously in the same formulation; administered in separate formulations and within about 48 hours, within about 36 hours, within about 24 hours, within about 16 hours, within about 12 hours, within about 8 hours, within about 4 hours, within about 2 hours, within about 1 hour, within about 30 minutes, or within about 15 minutes or less) one or more additional therapeutic agents.

Ribavirin and Other Antiviral Agents

Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The invention also contemplates use of derivatives of ribavirin (see, e.g., U.S. Pat. No. 6,277,830). Ribavirin is administered in dosages of about 400, about 800, or about 1200 mg per day.

Other antiviral agents can be delivered in the treatment methods of the invention. For example, compounds that inhibit inosine monophosphate dehydrogenase (IMPDH) may have the potential to exert direct anti viral activity, and such compounds can be administered in combination with an IFN-α composition, as described herein. Drugs that are effective inhibitors of hepatitis C NS3 protease may be administered in combination with an IFN-α composition, as described herein. Hepatitis C NS3 protease inhibitors inhibit viral replication. Other agents such as inhibitors of HCV NS3 helicase are also attractive drugs for combinational therapy, and are contemplated for use in combination therapies described herein. Ribozymes such as Heptazymem and phosphorothioate oligonucleotides which are complementary to HCV protein sequences and which inhibit the expression of viral core proteins are also suitable for use in combination therapies described herein.

Liver Targeting Systems

Antiviral agents described herein can be targeted to the liver, using any known targeting means. Those skilled in the art are aware of a wide variety of compounds that have been demonstrated to target compounds to hepatocytes. Such liver targeting compounds include, but are not limited to, asialoglycopeptides; basic polyamino acids conjugated with galactose or lactose residues; galactosylated albumin; asialoglycoprotein-poly-L-lysine) conjugates; lactosaminated albumin; lactosylated albumin-poly-L-lysine conjugates; galactosylated poly-L-lysine; galactose-PEG-poly-L-lysine conjugates;. lactose-PEG-poly-L-lysine conjugates; asialofetuin; and lactosylated albumin.

In some embodiments, a liver targeting compound is conjugated directly to the antiviral agent. In other embodiments, a liver targeting compound is conjugated indirectly to the antiviral agent, e.g., via a linker. In still other embodiments, a liver targeting compound is associated with a delivery vehicle, e.g., a liposome or a microsphere, forming a hepatocyte targeted delivery vehicle, and the antiviral agent is delivered using the hepatocyte targeted delivery vehicle.

The terms “targeting to the liver” and “hepatocyte targeted” refer to targeting of an antiviral agent to a hepatocyte, such that at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, of the antiviral agent administered to the subject enters the liver via the hepatic portal and becomes associated with (e.g., is taken up by) a hepatocyte.

Combination Therapy with Two Different Interferon Receptor Agonists and Pirfenidone or Pirfenidone Analogs

As discussed above, the methods of the invention can be carried out using combinations of a Type I IFN receptor agonist and a Type II IFN receptor agonist; a Type I IFN receptor agonist and a Type III IFN receptor agonist; and a Type II IFN receptor agonist and a Type III IFN receptor agonist.

Type I IFN Receptor Agonist or Type III IFN Receptor Agonist; Type II IFN Receptor Agonist; and Pirfenidone or Pirfenidone Analog

IFN-γ can be administered in combination therapy with a Type I or a Type III IFN. IFN-γ is administered in an amount of about 25 μg to about 300 μg, or about 100 μg to about 200 μg, of drug per dose of IFN-γ, subcutaneously qd, qod, tiw, biw, qw, qow, three times per month, or monthly. In some embodiments, IFN-γ is administered with an IFN-α and pirfenidone or pirfenidone analog. Effective dosages of IFN-α generally range from about 3 μg/dose to about 300 μg/dose.

In one embodiment, the methods of the invention can be carried out by administering to the patient: 1) a dosage of INFERGEN®consensus IFN-α containing an amount of about 3 μg, about 9 μg, about 15 μg, about 18 μg, or about 27 μg, of drug per dose of INFERGEN®, subcutaneously qd, qod, tiw, biw, qw, qow, three times per month, once monthly, or per day substantially continuously or continuously; 2) a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 50 mg to about 5,000 mg, or about 400 mg to about 3600 mg, or about 800 mg to about 2400 mg, or about 1000 mg to about 1800 mg, or about 1200 mg to about 1600 mg, orally qd for the desired duration of IFN-α therapy; and 3) IFN-γ in an amount of about 25 μg to about 300 μg, or about 100 μg to about 200 μg, of drug per dose of IFN-γ, subcutaneously qd, qod, tiw, biw, qw, qow, three times per month, or monthly.

In another embodiment, the methods of the invention can be carried out by administering to the patient: 1) a dosage of IFN-α2a or IFN-α2b containing an amount of about 3 million Units (MU) to about 10 MU of drug per dose of IFN-α2a or IFN-α2b, subcutaneously qd, qod, tiw, biw, qw, qow, three times per month, once monthly, or per day substantially continuously or continuously; 2) a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α therapy; and 3) IFN-γ in an amount of about 25 μg to about 300 μg, or about 100 μg to about 200 μg, of drug per dose of IFN-γ, subcutaneously qd, qod, tiw, biw, qw, qow, three times per month, or monthly.

In another embodiment, the methods of the invention can be carried out by administering to the patient: 1) a dosage of PEGASYS®PEGylated IFN-α2a containing an amount of about 90 μg to about 180 μg, or about 135 μg, of drug per dose of PEGASYS®, subcutaneously qw qow, three times per month, or monthly; 2) a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α; and 3) IFN-γ in an amount of about 25 μg to about 300 μg, or about 100 μg to about 200 μg, of drug per dose of IFN-γ, subcutaneously qd, qod, tiw, biw, qw, qow, three times per month, or monthly.

In another embodiment, the methods of the invention can be carried out by administering to the patient: 1) a dosage of PEG-INTRON®PEGylated IFN-α2b containing an amount of about 0.5 μg to about 1.5 μg of drug per kg body weight per dose of PEG-INTRON®, subcutaneously qw, qow, three times per month, or monthly; 2) a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α therapy; and 3) IFN-γ in an amount of about 25 μg to about 300 μg, or about 100 μg to about 200 μg, of drug per dose of IFN-γ, subcutaneously qd, qod, tiw, biw, qw, qow, three times per month, or monthly.

In another embodiment, the methods of the invention can be carried out by administering to the patient: 1) a dosage of PEGylated consensus interferon (PEG-CIFN) containing an amount of about 18 μg to about 90 μg, or about 27 μg to about 60 μg, or about 45 μg, of CIFN amino acid weight per dose of PEG-CIFN, subcutaneously qw, qow, three times per month, or monthly; 2) a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α therapy; and and 3) IFN-γ in an amount of about 25 μg to about 300 μg, or about 100 μg to about 200 μg, of drug per dose of IFN-γ, subcutaneously qd, qod, tiw, biw, qw, qow, three times per month, or monthly.

In another embodiment, the methods of the invention can be carried out by administering to the patient: 1) a dosage of IFN-β in a range of from 3 μg to about 300 μg; 2) a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α therapy; and and 3) IFN-γ in an amount of about 25 μg to about 300 μg, or about 100 μg to about 200 μg, of drug per dose of IFN-γ, subcutaneously qd, qod, tiw, biw, qw, qow, three times per month, or monthly.

In another embodiment, the methods of the invention can be carried out by administering to the patient: 1) a dosage of a Type III IFN in a range of from about 3 μg/dose to about 300 μg/dose; 2) a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α therapy; and and 3) IFN-γ in an amount of about 25 μg to about 300 μg, or about 100 μg to about 200 μg, of drug per dose of IFN-γ, subcutaneously qd, qod, tiw, biw, qw, qow, three times per month, or monthly.

In another embodiment, the methods of the invention can be carried out by administering to the patient: 1) a dosage of a IFN-tau in a range of from about 3 μg/dose to about 300 μg/dose; 2) a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α therapy; and and 3) IFN-γ in an amount of about 25 μg to about 300 μg, or about 100 μg to about 200 μg, of drug per dose of IFN-γ, subcutaneously qd, qod, tiw, biw, qw, qow, three times per month, or monthly.

In another embodiment, the methods of the invention can be carried out by administering to the patient: 1) a dosage of a IFN-ω in a range of from about 3 μg/dose to about 300 μg/dose; 2) a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α therapy; and and 3) IFN-γ in an amount of about 25 μg to about 300 μg, or about 100 μg to about 200 μg, of drug per dose of IFN-γ, subcutaneously qd, qod, tiw, biw, qw, qow, three times per month, or monthly.

Type I IFN; Type III IFN; and Pirfenidone or Pirfenidone Analog

In some embodiments, the above-described methods are carried out by administering an effective dosage of a Type I interferon; an effective dosage of a Type III interferon; and an effective dosage of pirfenidone or a pirfenidone analog. Effective dosages of a Type I IFN generally range from about 3 μg/dose to about 300 μg/dose. Effective dosages of a Type III IFN generally range from about 3 μg/dose to about 300 μg/dose.

In one embodiment, the methods of the invention can be carried out by administering to the patient: 1) a dosage of INFERGEN®consensus IFN-α containing an amount of about 3 μg, about 9 μg, about 15 μg, about 18 μg, or about 27 μg, of drug per dose of INFERGEN®, subcutaneously qd, qod, tiw, biw, qw, qow, three times per month, once monthly, or per day substantially continuously or continuously; 2) a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α therapy; and 3) a dosage of a Type III IFN in a range of from about 3 μg/dose to about 300 μg/dose by subcutaneous or intramuscular injection, or by continuous delivery qd, qod, tiw, biw, qw, qow, three times per month, or monthly.

In another embodiment, the methods of the invention can be carried out by administering to the patient: 1) a dosage of IFN-α2a or IFN-α2b containing an amount of about 3 million Units (MU) to about 10 MU of drug per dose of IFN-α2a or IFN-α2b, subcutaneously qd, qod, tiw, biw, qw, qow, three times per month, once monthly, or per day substantially continuously or continuously; 2) a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α therapy; and 3) a dosage of a Type III IFN in a range of from about 3 μg/dose to about 300 μg/dose by subcutaneous or intramuscular injection, or by continuous delivery qd, qod, tiw, biw, qw, qow, three times per month, or monthly.

In another embodiment, the methods of the invention can be carried out by administering to the patient: 1) a dosage of PEGASYS®PEGylated IFN-α2a containing an amount of about 90 μg to about 180 μg, or about 135 μg, of drug per dose of PEGASYS®, subcutaneously qw qow, three times per month, or monthly; 2) a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α; and 3) a dosage of a Type III IFN in a range of from about 3 jig/dose to about 300 μg/dose by subcutaneous or intramuscular injection, or by continuous delivery qd, qod, tiw, biw, qw, qow, three times per month, or monthly.

In another embodiment, the methods of the invention can be carried out by administering to the patient: 1) a dosage of PEG-INTRON®PEGylated IFN-α2b containing an amount of about 0.5 μg to about 1.5 μg of drug per kg body weight per dose of PEG-INTRON®, subcutaneously qw, qow, three times per month, or monthly; 2) a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α therapy; and 3) a dosage of a Type III IFN in a range of from about 3 μg/dose to about 300 μg/dose by subcutaneous or intramuscular injection, or by continuous delivery qd, qod, tiw, biw, qw, qow, three times per month, or monthly.

In another embodiment, the methods of the invention can be carried out by administering to the patient: 1) a dosage of PEGylated consensus interferon (PEG-CIFN) containing an amount of about 18 μg to about 90 μg, or about 27 μg to about 60 μg, or about 45 μg, of CIFN amino acid weight per dose-of PEG-CIFN, subcutaneously qw, qow, three times per month, or monthly; 2) a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α therapy; and and 3) a dosage of a Type III IFN in a range of from about 3 μg/dose to about 300 μg/dose by subcutaneous or intramuscular injection, or by continuous delivery qd, qod, tiw, biw, qw, qow, three times per month, or monthly.

In another embodiment, the methods of the invention can be carried out by administering to the patient: 1) a dosage of IFN-β in a range of from 3 μg to about 300 μg; 2) a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α therapy; and and 3) a dosage of a Type III IFN in a range of from about 3 μg/dose to about 300 μg/dose by subcutaneous or intramuscular injection, or by continuous delivery qd, qod, tiw, biw, qw, qow, three times per month, or monthly.

In another embodiment, the methods of the invention can be carried out by administering to the patient: 1) a dosage of a IFN-tau in a range of from about 3 μg/dose to about 300 μg/dose; 2) a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α therapy; and and 3) a dosage of a Type III IFN in a range of from about 3 μg/dose to about 300 μg/dose by subcutaneous or intramuscular injection, or by continuous delivery qd, qod, tiw, biw, qw, qow, three times per month, or monthly.

In another embodiment, the methods of the invention can be carried out by administering to the patient: 1) a dosage of a IFN-ω in a range of from about 3 μg/dose to about 300 μg/dose; 2) a weight-based dosage of pirfenidone or a specific pirfenidone analog in the range of about 5 mg/kg of body weight to about 125 mg/kg of body weight, or a fixed dosage of pirfenidone or a specific pirfenidone analog in the range of about 400 mg to about 3600 mg, or about 800 mg to about 2400 mg, or about 1000 mg to about 1800 mg, or about 1200 mg to about 1600 mg, orally qd for the desired duration of IFN-α therapy; and and 3) a dosage of a Type III IFN in a range of from about 3 μg/dose to about 300 μg/dose by subcutaneous or intramuscular injection, or by continuous delivery qd, qod, tiw, biw, qw, qow, three times per month, or monthly.

Determining Effectiveness of Treatment

Whether a subject method is effective in treating a hepatitis virus infection, particularly an HCV infection, can be determined by measuring viral load, or by measuring a parameter associated with HCV infection, including, but not limited to, liver fibrosis.

Viral load can be measured by measuring the titer or level of virus in serum. These methods include, but are not limited to, a quantitative polymerase chain reaction (PCR) and a branched DNA (bDNA) test. For example, quantitative assays for measuring the viral load (titer) of HCV RNA have been developed. Many such assays are available commercially, including a quantitative reverse transcription PCR (RT-PCR) (Amplicor HCV Monitor™, Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329.

As noted above, whether a subject method is effective in treating a hepatitis virus infection, e.g., an HCV infection, can be determined by measuring a parameter associated with hepatitis virus infection, such as liver fibrosis. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

As one non-limiting example, levels of serum alanine aminotransferase (ALT) are measured, using standard assays. In general, an ALT level of less than about 45 international units per milliliter serum is considered normal. In some embodiments, an effective amount of IFNα is an amount effective to reduce ALT levels to less than about 45 IU/ml serum.

Subjects Suitable for Treatment

Individuals who have been clinically diagnosed as infected with an alphavirus are suitable for treatment with a method of the instant invention. Of particular interest in some embodiments are individuals who have been clinically diagnosed as infected with WNV. Of particular interest in other embodiments are individuals who have been clinically diagnosed as infected with a hepatitis virus.

Of particular interest in some embodiments are individuals who have been clinically diagnosed as infected with a hepatitis virus (e.g., HAV, HBV, HCV, delta, etc.), particularly HCV. Such individuals are suitable for treatment with a method of the instant invention. Individuals who are infected with HCV are identified as having HCV RNA in their blood, and/or having anti-HCV antibody in their serum. Such individuals include naive individuals (e.g., individuals not previously treated for HCV, particularly those who have not previously received IFN-α-based or ribavirin-based therapy) and individuals who have failed prior treatment for HCV (“treatment failure” patients). Treatment failure patients include non-responders (e.g., individuals in whom the HCV titer was not significantly or sufficiently reduced by a previous treatment for HCV, particularly a previous IFN-α monotherapy using a single form of IFN-α); and relapsers (e.g., individuals who were previously treated for HCV (particularly a previous IFN-α monotherapy using a single form of IFN-α), whose HCV titer decreased significantly, and subsequently increased). In particular embodiments of interest, individuals have an HCV titer of at least about 10⁵, at least about 5×10⁵, or at least about 10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1 and particular HCV subtypes and quasispecies.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### IFN-α and Pirfenidone Inhibit Viral Growth

Materials and Methods

The following experiments were carried out using the standard cytopathic effect (CPE) assay as described by Ozes et al. (Ozes O N, Reiter Z, Klein S, Blatt L M, Taylor M W. A comparison of interferon-Con1 with natural recombinant interferons-alpha: antiviral, antiproliferative, and natural killer-inducing activities. J Interferon Res 1992 Feb.;12(1):55-9)

The cell line used was HeLa. The virus used was VSV. The results indicated that low doses of Pirfenidone enhance the antiviral effects of interferon.

Various amounts of interferon (e.g., 19 ng, 4.8 ng, 1.2 ng, 0.3 ng, 0.076 ng, 0.019 ng, 0.0049 ng, or 0.001 ng) was added to culture medium along with 0 μg, 3 μg, 30 μg, or 300 μg pirfenidone (PD); and the antiviral effect was determined.

Results

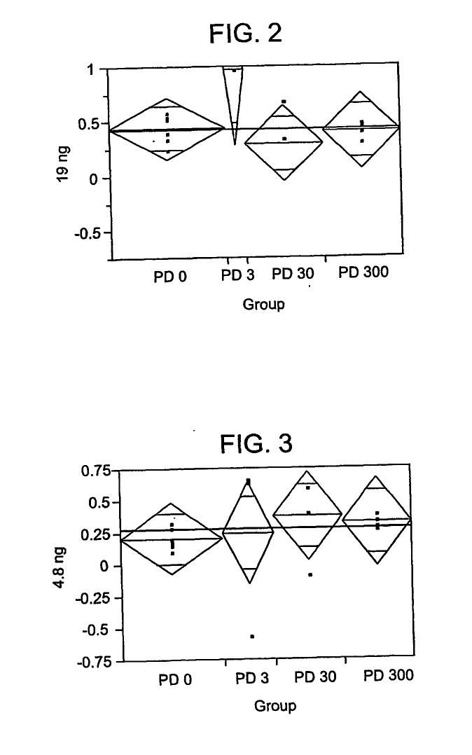

The results for 19 ng are shown in FIG. 2, and Tables 2-4.

TABLE 2
Oneway Anova
Summary of Fit

| | |
|---|---|
| Rsquare | 0.257142 |
| Adj Rsquare | 0.054544 |
| Root Mean Square Error | 0.31376 |
| Mean of Response | 0.4308 |
| Observations (or Sum Wgts) | 15 |

TABLE 3
Analysis of Variance

| Source | DF | Sum of Squares | Mean Square | F Ratio | Prob > F |
|---|---|---|---|---|---|
| Group | 3 | 0.3748473 | 0.124949 | 1.2692 | 0.3327 |
| Error | 11 | 1.0828971 | 0.098445 | | |
| C. Total | 14 | 1.4577444 | | | |

TABLE 4
Means for Oneway Anova

| Level | Number | Mean | Std Error | Lower 95% | Upper 95% |
|---|---|---|---|---|---|
| PD 0 | 6 | 0.442333 | 0.12809 | 0.1604 | 0.7243 |
| PD 3 | 1 | 0.979000 | 0.31376 | 0.2884 | 1.6696 |
| PD 30 | 4 | 0.296750 | 0.15688 | −0.0485 | 0.6420 |
| PD 300 | 4 | 0.410500 | 0.15688 | 0.0652 | 0.7558 |

Std Error uses a pooled estimate of error variance

The results for 4.8 ng are shown in FIG. 3, and Tables 5-7.

TABLE 5
Oneway Anova
Summary of Fit

| | |
|---|---|
| Rsquare | 0.056195 |
| Adj Rsquare | −0.16161 |
| Root Mean Square Error | 0.320506 |
| Mean of Response | 0.281529 |
| Observations (or Sum Wgts) | 17 |

TABLE 6
Analysis of Variance

| Source | DF | Sum of Squares | Mean Square | F Ratio | Prob > F |
|---|---|---|---|---|---|
| Group | 3 | 0.0795115 | 0.026504 | 0.2580 | 0.8543 |
| Error | 13 | 1.3354127 | 0.102724 | | |
| C. Total | 16 | 1.4149242 | | | |

TABLE 7
Means for Oneway Anova

| Level | Number | Mean | Std Error | Lower 95% | Upper 95% |
|---|---|---|---|---|---|
| PD 0 | 6 | 0.208000 | 0.13085 | −0.0747 | 0.49068 |
| PD 3 | 3 | 0.245000 | 0.18504 | −0.1548 | 0.64476 |
| PD 30 | 4 | 0.375750 | 0.16025 | 0.0295 | 0.72196 |
| PD 300 | 4 | 0.325000 | 0.16025 | −0.0212 | 0.67121 |

Std Error uses a pooled estimate of error variance

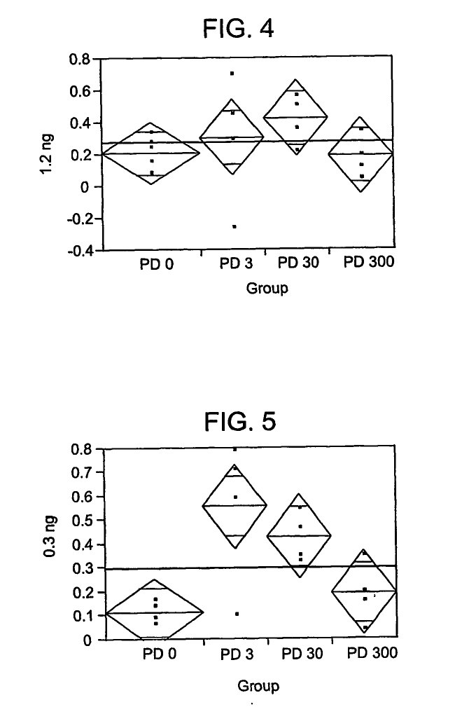

The results for 1.2 ng are shown in FIG. 4, and Tables 8-10.

TABLE 8
Oneway Anova
Summary of Fit

| | |
|---|---|
| Rsquare | 0.181922 |
| Adj Rsquare | 0.00662 |
| Root Mean Square Error | 0.219734 |
| Mean of Response | 0.273722 |
| Observations (or Sum Wgts) | 18 |

TABLE 9
Analysis of Variance

| Source | DF | Sum of Squares | Mean Square | F Ratio | Prob > F |
|---|---|---|---|---|---|
| Group | 3 | 0.15031928 | 0.050106 | 1.0378 | 0.4062 |
| Error | 14 | 0.67596433 | 0.048283 | | |
| C. Total | 17 | 0.82628361 | | | |

TABLE 10
Means for Oneway Anova

| Level | Number | Mean | Std Error | Lower 95% | Upper 95% |
|---|---|---|---|---|---|
| PD 0 | 6 | 0.209167 | 0.08971 | 0.0168 | 0.40157 |
| PD 3 | 4 | 0.306000 | 0.10987 | 0.0704 | 0.54164 |
| PD 30 | 4 | 0.424750 | 0.10987 | 0.1891 | 0.66039 |
| PD 300 | 4 | 0.187250 | 0.10987 | −0.0484 | 0.42289 |

Std Error uses a pooled estimate of error variance

The results for 0.3 ng are shown in FIG. 5, and Tables 11-13.

TABLE 11
Oneway Anova
Summary of Fit

| | |
|---|---|
| Rsquare | 0.610176 |
| Adj Rsquare | 0.526642 |
| Root Mean Square Error | 0.163475 |
| Mean of Response | 0.299833 |
| Observations (or Sum Wgts) | 18 |

TABLE 12
Analysis of Variance

| Source | DF | Sum of Squares | Mean Square | F Ratio | Prob > F |
|---|---|---|---|---|---|
| Group | 3 | 0.58561892 | 0.195206 | 7.3045 | 0.0035 |
| Error | 14 | 0.37413558 | 0.026724 | | |
| C. Total | 17 | 0.95975450 | | | |

TABLE 13
Means for Oneway Anova

| Level | Number | Mean | Std Error | Lower 95% | Upper 95% |
|---|---|---|---|---|---|
| PD 0 | 6 | 0.112333 | 0.06674 | −0.0308 | 0.25547 |
| PD 3 | 4 | 0.556250 | 0.08174 | 0.3809 | 0.73156 |
| PD 30 | 4 | 0.429750 | 0.08174 | 0.2544 | 0.60506 |
| PD 300 | 4 | 0.194750 | 0.08174 | 0.0194 | 0.37006 |

Std Error uses a pooled estimate of error variance

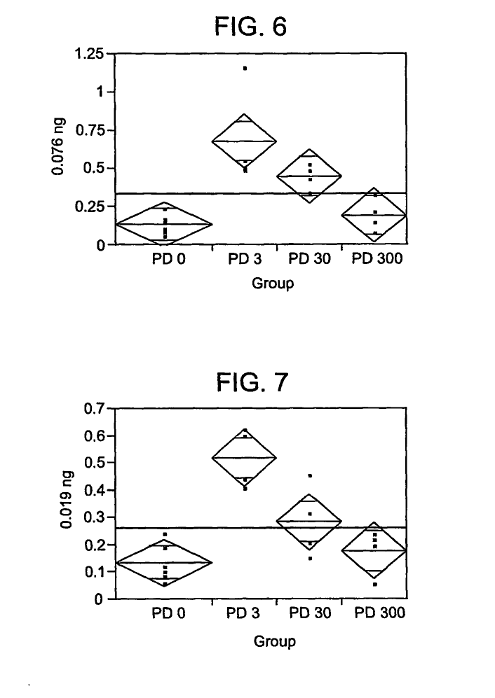

The results for 0.076 ng are shown in FIG. 6 and Tables 14-16.

TABLE 14
Oneway Anova
Summary of Fit

| | |
|---|---|
| Rsquare | 0.682855 |
| Adj Rsquare | 0.614896 |
| Root Mean Square Error | 0.167214 |
| Mean of Response | 0.340111 |
| Observations (or Sum Wgts) | 18 |

TABLE 15
Analysis or Variance

| Source | DF | Sum of Squares | Mean Square | F Ratio | Prob > F |
|---|---|---|---|---|---|
| Group | 3 | 0.8428345 | 0.280945 | 10.0480 | 0.0009 |
| Error | 14 | 0.3914453 | 0.027960 | | |
| C. Total | 17 | 1.2342798 | | | |

TABLE 16
Means for Oneway Anova

| Level | Number | Mean | Std Error | Lower 95% | Upper 95% |
|---|---|---|---|---|---|
| PD 0 | 6 | 0.137500 | 0.06826 | −0.0089 | 0.28391 |
| PD 3 | 4 | 0.680000 | 0.08361 | 0.5007 | 0.85932 |
| PD 30 | 4 | 0.450500 | 0.08361 | 0.2712 | 0.62982 |
| PD 300 | 4 | 0.193750 | 0.08361 | 0.0144 | 0.37307 |

Std Error uses a pooled estimate of error variance

The results for 0.019 ng are shown in FIG. 7 and Tables 17-19.

TABLE 17
Oneway Anova
Summary of Fit

| | |
|---|---|
| Rsquare | 0.746371 |
| Adj Rsquare | 0.692022 |
| Root Mean Square Error | 0.097391 |
| Mean of Response | 0.263111 |
| Observations (or Sum Wgts) | 18 |

TABLE 18
Analysis of Variance

| Source | DF | Sum of Squares | Mean Square | F Ratio | Prob > F |
|---|---|---|---|---|---|
| Group | 3 | 0.39077278 | 0.130258 | 13.7329 | 0.0002 |
| Error | 14 | 0.13279100 | 0.009485 | | |
| C. Total | 17 | 0.52356378 | | | |

TABLE 19
Means for Oneway Anova

| Level | Number | Mean | Std Error | Lower 95% | Upper 95% |
|---|---|---|---|---|---|
| PD 0 | 6 | 0.135000 | 0.03976 | 0.04972 | 0.22028 |
| PD 3 | 4 | 0.519000 | 0.04870 | 0.41456 | 0.62344 |
| PD 30 | 4 | 0.284000 | 0.04870 | 0.17956 | 0.38844 |
| PD 300 | 4 | 0.178500 | 0.04870 | 0.07406 | 0.28294 |

Std Error uses a pooled estimate of error variance

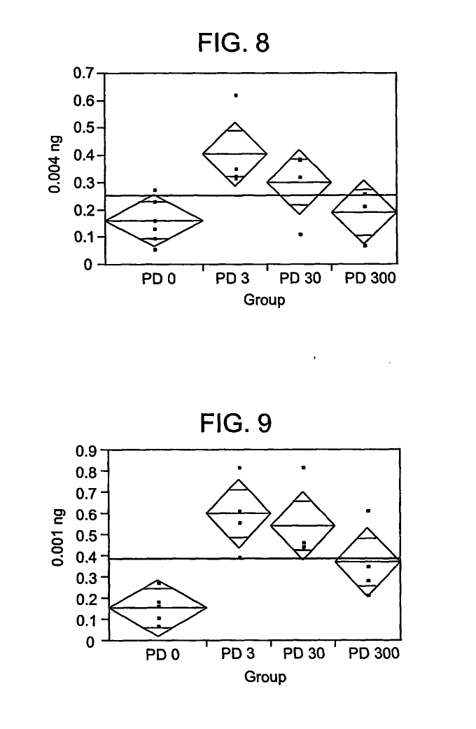

The results for 0.0049 ng are shown in FIG. 8 and Tables 20-22.

TABLE 20
Oneway Anova
Summary of Fit

| | |
|---|---|
| Rsquare | 0.501034 |
| Adj Rsquare | 0.394113 |
| Root Mean Square Error | 0.109586 |
| Mean of Response | 0.254667 |
| Observations (or Sum Wgts) | 18 |

TABLE 21
Analysis of Variance

| Source | DF | Sum of Squares | Mean Square | F Ratio | Prob > F |
|---|---|---|---|---|---|
| Group | 3 | 0.16882342 | 0.056274 | 4.6860 | 0.0181 |
| Error | 14 | 0.16812658 | 0.012009 | | |
| C. Total | 17 | 0.33695000 | | | |

TABLE 22
Means for Oneway Anova

| Level | Number | Mean | Std Error | Lower 95% | Upper 95% |
|---|---|---|---|---|---|
| PD 0 | 6 | 0.162833 | 0.04474 | 0.06688 | 0.25879 |
| PD 3 | 4 | 0.407000 | 0.05479 | 0.28948 | 0.52452 |
| PD 30 | 4 | 0.303250 | 0.05479 | 0.18573 | 0.42077 |
| PD 300 | 4 | 0.191500 | 0.05479 | 0.07398 | 0.30902 |

Std Error uses a pooled estimate of error variance

The results for 0.001 ng are shown in FIG. 9 and Tables 23-25.

TABLE 23
Oneway Anova
Summary of Fit

| | |
|---|---|
| Rsquare | 0.656429 |
| Adj Rsquare | 0.582806 |
| Root Mean Square Error | 0.150003 |
| Mean of Response | 0.388222 |
| Observations (or Sum Wgts) | 18 |

TABLE 24
Analysis of Variance

| Source | DF | Sum of Squares | Mean Square | F Ratio | Prob > F |
|---|---|---|---|---|---|
| Group | 3 | 0.60186186 | 0.200621 | 8.9162 | 0.0015 |
| Error | 14 | 0.31501125 | 0.022501 | | |
| C. Total | 17 | 0.91687311 | | | |

TABLE 25
Means for Oneway Anova

| Level | Number | Mean | Std Error | Lower 95% | Upper 95% |
|---|---|---|---|---|---|
| PD 0 | 6 | 0.155500 | 0.06124 | 0.02416 | 0.28684 |
| PD 3 | 4 | 0.600250 | 0.07500 | 0.43939 | 0.76111 |
| PD 30 | 4 | 0.543000 | 0.07500 | 0.38214 | 0.70386 |
| PD 300 | 4 | 0.370500 | 0.07500 | 0.20964 | 0.53136 |

Std Error uses a pooled estimate of error variance

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of treating a hepatitis virus infection in an individual, the method comprising administering to the individual an effective amount of interferon alpha (IFN-α) and an effective amount of pirfenidone or a pirfenidone analog.

2. The method of claim 1, wherein a sustained viral response is achieved.

3. The method of claim 1, wherein the IFN-α is consensus interferon.

4. The method of claim 1, wherein the IFN-α is selected from IFN-α2a, IFN-α2b, and IFN-α2c.

5. A method of treating an alphaviral infection in an individual, the method comprising administering to the individual an effective amount of an interferon-alpha (IFN-α) and an effective amount of pirfenidone or a pirfenidone analog.

6. The method of claim 5, wherein the method comprises administering to the individual a synergistically effective amount of IFN-α and a pirfenidone analog.

7. The method of claim 5, wherein the method comprises administering to the individual an effective amount of IFN-α and an amount of pirfenidone or a pirfenidone analog effective to reduce the incidence or severity of side effects ordinarily experienced by the individual in respones to IFN-α monotherapy for treatment of alphaviral infection.

8. The method of claim 7, wherein the amount of IFN-α administered to the individual is at least about 90% of the maximum tolerated dose (MTD) of the patient for IFN-α in the context of IFN-α monotherapy for treatment of the alphaviral infection.

9. The method of claim 8, wherein the amount of IFN-α is at least about 100% of the MID.

10. The method of any one of claims 5-9, wherein the alphaviral infection is a hepatitis viral infection.

11. The method of claim 10, wherein the hepatitis viral infection is a hepatitis C viral infection.

12. The method of any one of claims 5-9, wherein the alphaviral infection is a West Nile viral infection.


SEQUENCE LISTING (1 sequence)

SEQ ID NO: 1 - protein, 167 aa, Artificial Sequence
  consensus sequence
MCDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNVDSILAVKKYFQRITLYLTEKKYSPCAWEVVRAEIMRSFSLSTNLQERLRRKE